(19) United States
(12) Patent Application Publication
(10) Pub. No.: US 2008/0075728 A1
(43) Pub. Date: Mar. 27, 2008

(54) Combination Therapies Of Hmgb And Complement Inhibitors Against Inflammation

(76) Inventor: Walter Newman, Boston, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/632,257

(22) PCT Filed: Jul. 20, 2005
(86) PCT No.: PCT/US05/25799

§ 371 Date: Mar. 6, 2007

Related U.S. Application Data
(60) Provisional application No. 60/589,608, filed on Jul. 20, 2004.

(51) Int. Cl. A61K 39/395; A61K 31/22; A61K 38/02; A61P 1/18; A61P 11/06; A61P 17/06; A61P 25/00; A61P 29/00; A61P 19/02; A61P 17/02; A61P 11/00; A61P 1/00; A61K 38/17

(52) U.S. Cl. 424/152.1; 424/172.1; 514/12; 514/2; 514/546

(57) ABSTRACT

Compositions and methods are disclosed for treating a condition characterized by activation of an inflammatory cytokine cascade in a patient. The compositions comprise an HMGB A box and an inhibitor of complement biological activity, and/or an antibody that binds an HMGB polypeptide or biologically active fragment thereof and an inhibitor of complement biological activity, and/or an inhibitor of HMGB receptor binding and an inhibitor of complement biological activity. The methods comprise treating a cell or a patient with sufficient amounts of the composition to inhibit the release of proinflammatory cytokine(s) and/or inhibit the inflammatory cytokine cascade.

### RELATED APPLICATION

This application claims the benefit of U.S. Provisional Application No. 60/589,608, filed Jul. 20, 2004, the entire teachings of which are incorporated herein by reference.

### BACKGROUND OF THE INVENTION

Inflammation is often induced by proinflammatory cytokines, such as tumor necrosis factor (TNF), interleukin (IL)-1α, IL-1β, IL-6, macrophage migration inhibitory factor (MIF), and other compounds. These proinflammatory cytokines are produced by several different cell types, including immune cells (for example, monocytes, macrophages and neutrophils) and non-immune cells, such as fibroblasts, osteoblasts, smooth muscle cells, epithelial cells, and neurons. These proinflammatory cytokines contribute to various disorders during the early stages of an inflammatory cytokine cascade.

The early proinflammatory cytokines (e.g., TNF, IL-1, etc.) mediate inflammation, and induce the late release of high mobility group box 1 (HMGB1; also known as HMG-1 and HMG1), a protein that accumulates in serum and mediates delayed lethality and further induction of early proinflammatory cytokines. HMGB1 was first identified as the founding member of a family of DNA-binding proteins termed high mobility group box (HMGB) proteins that are critical for DNA structure and stability. It was identified as a ubiquitously expressed nuclear protein that binds double-stranded DNA without sequence specificity. The HMGB1 molecule has three domains: two DNA binding motifs termed HMGB A and HMGB B boxes, and an acidic carboxyl terminus. The two HMGB boxes are highly conserved 80 amino acid, L-shaped domains.

Recent evidence has implicated HMGB1 as a cytokine mediator of a number of inflammatory conditions. In addition, fragments of HMGB are also thought to modulate inflammation. The delayed kinetics of HMGB1 appearance during endotoxemia make it a potentially good therapeutic target in the treatment of inflammatory conditions.

### SUMMARY OF THE INVENTION

The present invention is based on the discoveries that combination therapies involving agents that inhibit HMGB biological activity and agents that inhibit complement biological activity can be used for the treatment of inflammatory conditions. Agents that inhibit HMGB biological activity include the HMGB A box, antibodies to HMGB (e.g., antibodies to the HMGB B box, antibodies to the HMGB A box) and inhibitors of HMGB receptor binding and/or HMGB signaling.

Accordingly, in one embodiment, the invention is a pharmaceutical composition comprising an agent that inhibits HMGB biological activity and an agent that inhibits complement biological activity. In one embodiment, the invention is a pharmaceutical composition comprising an inhibitor of HMGB receptor binding and/or HMGB signaling and an agent that inhibits complement biological activity. Agents that inhibit HMGB receptor binding and/or signaling include, e.g., polypeptides comprising a high mobility group box (HMGB) A box, antibodies to HMGB and/or HMGB boxes (e.g., A boxes, B boxes) or antigen-binding fragments thereof, HMGB small molecule antagonists, antibodies to TLR2 or antigen-binding fragments thereof, soluble TLR2 polypeptides, TLR2 small molecule antagonists, TLR2 dominant mutant proteins, antibodies to TLR4 or antigen-binding fragments thereof, soluble TLR4 polypeptides, TLR4 small molecule antagonists, TLR4 dominant mutant proteins, antibodies to RAGE or antigen-binding fragments thereof, soluble RAGE, RAGE small molecule antagonists and RAGE dominant mutant proteins. In one embodiment, the inhibitor of HMGB receptor binding is an inhibitor of HMGB1 receptor binding.

In another embodiment, the invention is a pharmaceutical composition comprising a polypeptide comprising a high mobility group box (HMGB) A box or a biologically active fragment thereof and an agent that inhibits complement biological activity.

In another embodiment, the invention is a pharmaceutical composition comprising an antibody or an antigen-binding fragment thereof and an agent that inhibits complement biological activity, wherein the antibody or antigen-binding fragment binds an HMGB polypeptide or a fragment thereof (e.g., an HMGB B box polypeptide or biologically active fragment thereof, an HMGB A box polypeptide or biologically active fragment thereof).

In another embodiment, the invention is a method of treating an inflammatory condition in a patient comprising administering to the patient a composition comprising an agent that inhibits HMGB biological activity and an agent that inhibits complement biological activity. In one embodiment, the composition that is administered comprises an inhibitor of HMGB receptor binding and/or HMGB signaling and an agent that inhibits complement biological activity.

In yet another embodiment, the invention is a method of treating an inflammatory condition in a patient comprising administering to the patient a composition comprising a polypeptide comprising a high mobility group box (HMGB) A box or a biologically active fragment thereof and an agent that inhibits complement biological activity.

In still another embodiment, the invention is a method of treating an inflammatory condition in a patient comprising administering to the patient a composition comprising an antibody or an antigen-binding fragment thereof and an agent that inhibits complement biological activity, wherein the antibody or antigen-binding fragment binds an HMGB polypeptide or a biologically active fragment thereof.

The present invention offers the advantage of providing individuals in need of treatment for inflammatory conditions new and effective combination therapy compositions.

### BRIEF DESCRIPTION OF THE DRAWINGS

FIG. 1A is the amino acid sequence of a human HMG1 polypeptide (SEQ ID NO:1).

FIG. 1B is the amino acid sequence of a rat and mouse HMG1 polypeptide (SEQ ID NO:2).

FIG. 1C is the amino acid sequence of a human HMG2 polypeptide (SEQ ID NO:3).

FIG. 1D is the amino acid sequence of a human, mouse, and rat HMG1 A box polypeptide (SEQ ID NO:4).

FIG. 1E is the amino acid sequence of a human, mouse, and rat HMG1 B box polypeptide (SEQ ID NO:5).

FIG. 2A is the nucleic acid sequence of HMG1L5 (formerly HMG1L10; SEQ ID NO:9), which encodes an HMGB polypeptide.

FIG. 2B is the polypeptide sequence of HMG1L5 (formerly HMG1L10; SEQ ID NO:10), which is encoded by the nucleic acid sequence of FIG. 2A.

FIG. 2C is the nucleic acid sequence of HMG1L1 (SEQ ID NO:11), which encodes an HMGB polypeptide.

FIG. 2D is the polypeptide sequence of HMG1L1 (SEQ ID NO:12), which is encoded by the nucleic acid sequence of FIG. 2C.

FIG. 2E is the nucleic acid sequence of HMG1L4 (SEQ ID NO:13), which encodes an HMGB polypeptide.

FIG. 2F is the polypeptide sequence of HMG1L4 (SEQ ID NO:14), which is encoded by the nucleic acid sequence of FIG. 2E.

FIG. 2G is the nucleic acid sequence of the BAC clone RP11-395A23 (SEQ ID NO:15), which encodes an HMG polypeptide sequence.

FIG. 2H is the amino acid sequence of the BAC clone RP11-395A23 (SEQ ID NO:16), which is encoded by the nucleic acid sequence of FIG. 2G.

FIG. 2I is the nucleic acid sequence of HMG1L9 (SEQ ID NO:17), which encodes an HMGB polypeptide.

FIG. 2J is the polypeptide sequence of HMG1L9 (SEQ ID NO:18), which is encoded by the nucleic acid sequence of FIG. 2I.

FIG. 2K is the nucleic acid sequence of LOC122441 (SEQ ID NO:19), which encodes an HMGB polypeptide.

FIG. 2L is the polypeptide sequence of LOC122441 (SEQ ID NO:20), which is encoded by the nucleic acid sequence of FIG. 2K.

FIG. 2M is the nucleic acid sequence of LOC139603 (SEQ ID NO:21), which encodes an HMGB polypeptide.

FIG. 2N is the polypeptide sequence of LOC139603 (SEQ ID NO:22), which is encoded by the nucleic acid sequence of FIG. 2M.

FIG. 2O is the nucleic acid sequence of HMG1L8 (SEQ ID NO:23), which encodes an HMGB polypeptide.

FIG. 2P is the polypeptide sequence of HMG1L8 (SEQ ID NO:24), which is encoded by the nucleic acid sequence of FIG. 2O.

FIG. 3 is a schematic representation of an overview of the complement activation pathways. Thick arrows indicate enzymatic or activating activity and thin arrows indicate reaction steps.

### DETAILED DESCRIPTION OF THE INVENTION

The present invention is based on the discovery that inhibitors of complement biological activity can be combined with agents that inhibit HMGB biological activity, to form pharmaceutical compositions (combination therapy compositions) for use in treating an inflammatory condition in a patient. As described herein, agents that inhibit HMGB biological activity include, e.g., the HMGB A box, antibodies to HMGB (e.g., antibodies to the HMGB B box, antibodies to the HMGB A box) and inhibitors of HMGB receptor binding and/or HMGB signaling.

A proinflammatory domain of HMGB (e.g., HMGB1) is the B box (and in particular, the first 20 amino acids of the B box), and antibodies that bind to the B box and inhibit proinflammatory cytokine release and inflammatory cytokine cascades can be used to alleviate deleterious symptoms caused by inflammatory cytokine cascades (PCT Publication No. WO 02/092004, the entire teachings of which are incorporated herein by reference). In addition, the A box is a weak agonist of inflammatory cytokine release, and competitively inhibits the proinflammatory activity of the B box and of HMGB (e.g., HMGB1) (PCT Publication No. WO 02/092004). Inhibitors of HMGB receptor binding and/or HMGB signaling (e.g., antibodies to HMGB (e.g., antibodies to HMGB B boxes, antibodies to HMGB A boxes) or antigen-binding fragments thereof, HMGB A box polypeptides, antibodies to RAGE or antigen-binding fragments thereof (e.g., as taught in U.S. Pat. Nos. 5,864,018 and 5,852,174), antibodies to TLR2 or antigen-binding fragments thereof (e.g., as taught in PCT Publication Nos. WO 01/36488 and WO 00/75358), soluble RAGE, soluble TLR2 (e.g., as taught in Iwaki et al., J. Biol. Chem. 277(27):24315-24320 (2002)), HMGB small molecule antagonists (e.g., ethyl pyruvate, certain derivatives of isoxazole, isoxazolidine, isothiazole and isothiazolidine compounds), RAGE small molecule antagonists (e.g., as taught in PCT Publication Nos. WO 01/99210, WO 02/06965 and WO 03/075921, and U.S. Published Application No. 2002/0193432A1), TLR2 small molecule antagonists, TLR2 dominant mutant proteins, and RAGE dominant mutant proteins) can also be used to alleviate deleterious symptoms caused by inflammatory cytokine cascades. Therefore, HMGB A boxes, antibodies to HMGB and biologically active fragments thereof (e.g., HMGB A boxes or biologically active fragments thereof, HMGB B boxes or biologically active fragments thereof), and/or inhibitors of HMGB receptor binding and/or HMGB signaling, can be combined with an inhibitor of complement biological activity to treat inflammatory conditions.

HMGB Polypeptides

As used herein, an “HMGB polypeptide” is polypeptide that has at least 60%, more preferably, at least 70%, 75%, 80%, 85%, or 90%, and most preferably at least 95%, sequence identity to a sequence selected from the group consisting of SEQ ID NO:1, SEQ ID NO:2, SEQ ID NO:3, and SEQ ID NO:6 (MGKGDPKKPT GKMSSYAFFV QTCREEHKKK HPDASVNFSE FSKKCSERWK TMSAKEKGKF EDMAKADKAR YEREMKTYIP PKGETKKKFK DPNAPKRLPS AFFLFCSEYR PKIKGEHPGL SIGDVAKKLG EMWNNTAADD KQPYEKKAAK LKEKYEKDIA AYRAKGKPDA AKKGVVKAEK SKKKKEEEED EEDEEDEEEE EDEEDEEDEE EDDDDE) (as determined, for example, using the BLAST program and parameters described herein) and increases inflammation and/or increases release of a proinflammatory cytokine from a cell. In one embodiment, the HMGB polypeptide has one of the above biological activities. Typically, the HMGB polypeptide has both of the above biological activities.

The term “polypeptide” refers to a polymer of amino acids, and not to a specific length; thus, peptides, oligopeptides and proteins are included within the definition of a polypeptide. Preferably, the HMGB polypeptide is a mammalian HMGB polypeptide, for example, a human HMGB1 polypeptide. Preferably, the HMGB polypeptide contains a B box DNA binding domain and/or an A box DNA binding domain and/or an acidic carboxyl terminus as described herein.

Other examples of HMGB polypeptides are described in GenBank Accession Numbers AAA64970, AAB08987, P07155, AAA20508, S29857, P09429, NP_{—}002119, CAA31110, SO2826, U00431, X67668, NP_{—}005333, NM_{—}016957, and J04179, the entire teachings of which are incorporated herein by reference. Additional examples of HMGB polypeptides include, but are not limited to mammalian HMG1 ((HMGB1) as described, for example, in GenBank Accession Number U51677), mouse HMG1 as described, for example, in GenBank Accession Number CAA55631.1, rat HMG1 as described, for example, in GenBank Accession Number NP_{—}037095.1, cow HMG1 as described, for example, in GenBank Accession Number CAA31284.1, HMG2 ((HMGB2) as described, for example, in GenBank Accession Number M83665), HMG-2A ((HMGB3, HMG-4) as described, for example, in GenBank Accession Numbers NM_{—}005342 and NP_{—}005333), HMG14 (as described, for example, in GenBank Accession Number P05114), HMG17 (as described, for example, in GenBank Accession Number X13546), HMG1 (as described, for example, in GenBank Accession Number L17131), and HMGY (as described, for example, in GenBank Accession Number M23618); nonmammalian HMG T1 (as described, for example, in GenBank Accession Number X02666) and HMG T2 (as described, for example, in GenBank Accession Number L32859) (rainbow trout); HMG-X (as described, for example, in GenBank Accession Number D30765) (Xenopus); HMG D (as described, for example, in GenBank Accession Number X71138) and HMG Z (as described, for example, in GenBank Accession Number X71139) (Drosophila); NHP10 protein (HMG protein homolog NHP 1) (as described, for example, in GenBank Accession Number Z48008) (yeast); non-histone chromosomal protein (as described, for example, in GenBank Accession Number O00479) (yeast); HMG ½ like protein (as described, for example, in GenBank Accession Number Z11540) (wheat, maize, soybean); upstream binding factor (UBF-1) (as described, for example, in GenBank Accession Number X53390); PMS1 protein homolog 1 (as described, for example, in GenBank Accession Number U13695); single-strand recognition protein (SSRP, structure-specific recognition protein) (as described, for example, in GenBank Accession Number M86737); the HMG homolog TDP-1 (as described, for example, in GenBank Accession Number M74017); mammalian sex-determining region Y protein (SRY, testis-determining factor) (as described, for example, in GenBank Accession Number X53772); fungal proteins: mat-1 (as described, for example, in GenBank Accession Number AB009451), ste 11 (as described, for example, in GenBank Accession Number X53431) and Mc 1; SOX 14 (as described, for example, in GenBank Accession Number AF107043) (as well as SOX 1 (as described, for example, in GenBank Accession Number Y13436), SOX 2 (as described, for example, in GenBank Accession Number Z31560), SOX 3 (as described, for example, in GenBank Accession Number X71135), SOX 6 (as described, for example, in GenBank Accession Number AF309034), SOX 8 (as described, for example, in GenBank Accession Number AF226675), SOX 10 (as described, for example, in GenBank Accession Number AJ001183), SOX 12 (as described, for example, in GenBank Accession Number X73039) and SOX 21 (as described, for example, in GenBank Accession Number AF107044); lymphoid specific factor (LEF-1) (as described, for example, in GenBank Accession Number X58636); T-cell specific transcription factor (TCF-1) (as described, for example, in GenBank Accession Number X59869); MTT1 (as described, for example, in GenBank Accession Number M62810); and SP100-HMG nuclear autoantigen (as described, for example, in GenBank Accession Number U36501). Other examples of HMGB polypeptides include those encoded by nucleic acid sequences having Genbank Accession Numbers AAH81839 (rat high mobility group box 1), NP_{—}990233 (chicken high mobility group box 1), AAN11319 (dog high mobility group B1), AAC27653 (mole high mobility group protein), P07746 (trout high mobility group-T protein), AAA58771 (trout HMG-1), AAQ97791 (zebra fish high-mobility group box 1), AAH01063 (human high-mobility group box 2), and P10103 (cow high mobility group protein 1).

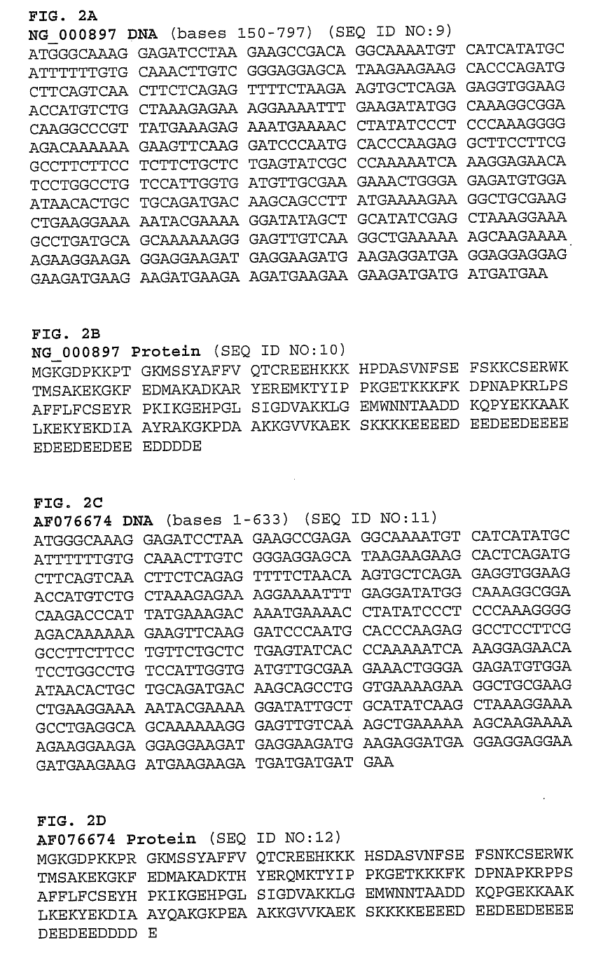

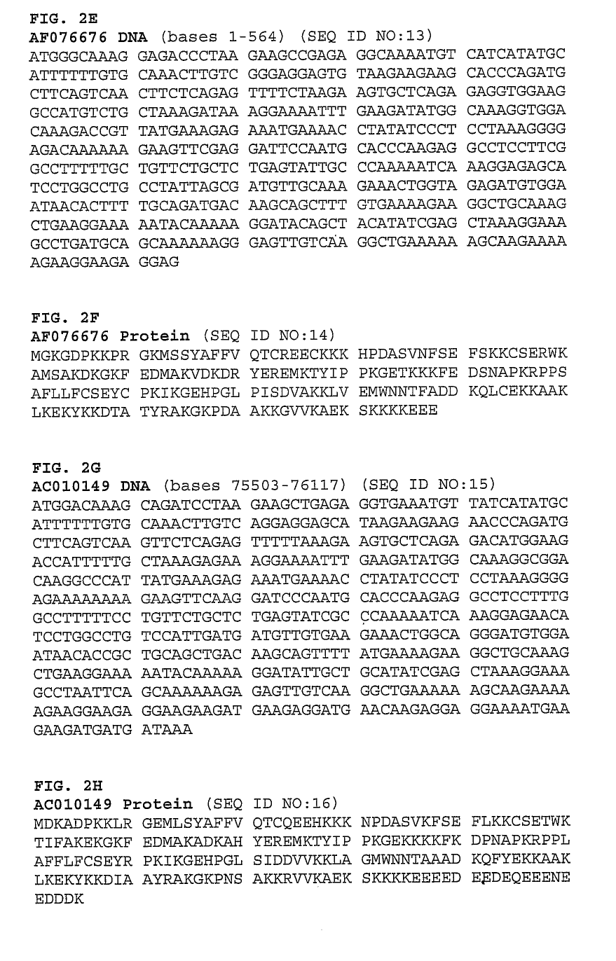

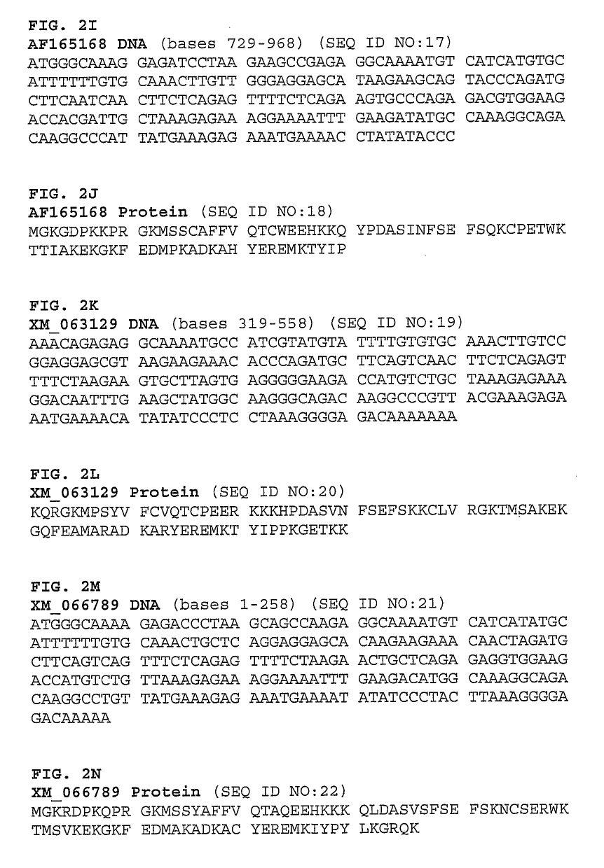

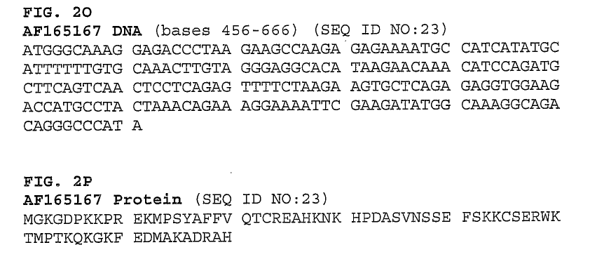

Other examples of HMGB proteins are polypeptides encoded by HMGB nucleic acid sequences having GenBank Accession Numbers NG_{—}000897 (HMG1L5) (and in particular by nucleotides 150-797 of NG_{—}000897, as shown in FIGS. 2A and 2B); AF076674 (HMG1L1) (and in particular by nucleotides 1-633 of AF076674, as shown in FIGS. 2C and 2D; AF076676 (HMG1L4) (and in particular by nucleotides 1-564 of AF076676, as shown in FIGS. 2E and 2F); AC010149 (HMG sequence from BAC clone RP11-395A23) (and in particular by nucleotides 75503-76117 of AC010149, as shown in FIGS. 2G and 2H); AF165168 (HMG1L9) (and in particular by nucleotides 729-968 of AF165168, as shown in FIGS. 2I and 2J); XM_{—}063129 (LOC122441) (and in particular by nucleotides 319-558 of XM_{—}063129, as shown in FIGS. 2K and 2L); XM_{—}066789 (LOC139603) (and in particular by nucleotides 1-258 of XM_{—}066789, as shown in FIGS. 2M and 2N); and AF165167 (HMG1L8) (and in particular by nucleotides 456-666 of AF165167, as shown in FIGS. 2O and 2P).

Optionally, the HMGB polypeptide is a substantially pure, or substantially pure and isolated, polypeptide that has been separated from components that naturally accompany it. As used herein, a polypeptide is said to be “isolated” or “purified” when it is substantially free of cellular material when it is isolated from recombinant and non-recombinant cells, or free of chemical precursors or other chemicals when it is chemically synthesized. A polypeptide, however, can be joined to another polypeptide with which it is not normally associated in a cell (e.g., in a “fusion protein”) and still be “isolated” or “purified.” It is understood, however, that preparations in which the polypeptide is not purified to homogeneity are useful. For example, the polypeptide may be in an unpurified form, for example, in a cell, cell milieu, or cell extract. The critical feature is that the preparation allows for the desired function of the polypeptide, even in the presence of considerable amounts of other components.

HMGB polypeptides can be purified from cells that naturally express it, purified from cells that have been altered to express it (recombinant), or synthesized using known protein synthesis methods. In one embodiment, the polypeptide is produced by recombinant DNA techniques. For example, a nucleic acid molecule encoding the polypeptide is cloned into an expression vector, the expression vector is introduced into a host cell and the polypeptide is expressed in the host cell. The polypeptide can then be isolated from the cells by an appropriate purification scheme using standard protein purification techniques.

Functional equivalents of HMGB (proteins or polypeptides that have one or more of the biological activities of an HMGB polypeptide) can also be used in the combination therapy compositions and methods of the present invention. Biologically active fragments, sequence variants, post-translationally modified proteins, and chimeric or fusion proteins comprising HMGB, a biologically active fragment or a variant are examples of functional equivalents of a protein. Variants include a substantially homologous polypeptide encoded by the same genetic locus in an organism, i.e., an allelic variant, as well as other splicing variants. Variants also encompass polypeptides derived from other genetic loci in an organism, but having substantial homology to the protein of interest, for example, an HMGB protein as described herein.

A variant polypeptide can differ in amino acid sequence by one or more substitutions, deletions, insertions, inversions, fusions, and truncations, or a combination of any of these. Further, variant polypeptides can be fully functional or can lack function in one or more activities. Fully functional variants typically contain only conservative variation or variation in non-critical residues or in non-critical regions. Functional variants can also contain substitution of similar amino acids that result in no change or an insignificant change in function. Alternatively, such substitutions may positively or negatively affect function to some degree.

Amino acids that are essential for function can be identified by methods known in the art, such as site-directed mutagenesis or alanine-scanning mutagenesis (Cunningham et al., Science, 244:1081-1085, 1989). The latter procedure introduces single alanine mutations at every residue in the molecule. The resulting mutant molecules are then tested for biological activity in vitro. Sites that are critical for polypeptide activity can also be determined by structural analysis such as crystallization, nuclear magnetic resonance or photoaffinity labeling (Smith et al., J. Mol. Biol., 224:899-904, 1992; and de Vos et al., Science, 255:306-312, 1992).

HMGB functional equivalents also include polypeptide fragments of HMGB. Fragments can be derived from an HMGB polypeptide or HMGB variant. As used herein, a fragment comprises at least 6 contiguous amino acids from an HMGB polypeptide. Useful fragments include those that retain one or more of the biological activities of the polypeptide. Examples of HMGB biologically active fragments include the B box, as well as biologically active fragments of the B box, for example, the first 20 amino acids of the B box (e.g., the first 20 amino acids of SEQ ID NO:5 (SEQ ID NO:44; NAPKRPPSAF FLFCSEYRPK) or SEQ ID NO:8 (SEQ ID NO:45; FKDPNAPKRL PSAFFLFCSE)).

Biologically active fragments (peptides which are, for example, 6, 9, 12, 15, 16, 20, 30, 35, 36, 37, 38, 39, 40, 50, 100 or more amino acids in length) can comprise a domain, segment, or motif that has been identified by analysis of the polypeptide sequence using well-known methods, e.g., signal peptides, extracellular domains, one or more transmembrane segments or loops, ligand binding regions, zinc finger domains, DNA binding domains, or post-translation modification sites. Example of domains include the A box and B box, as described herein.

Fragments can be discrete (not fused to other amino acids or polypeptides) or can be within a larger polypeptide. Further, several fragments can be comprised within a single larger polypeptide. In one embodiment a fragment designed for expression in a host can have heterologous pre- and pro-polypeptide regions fused to the amino terminus of the polypeptide fragment and an additional region fused to the carboxyl terminus of the fragment.

The invention also provides uses and methods for chimeric or fusion polypeptides containing an HMGB polypeptide or a functional equivalent of HMGB. These chimeric proteins comprise an HMGB polypeptide or fragment thereof operatively linked to a heterologous protein or polypeptide having an amino acid sequence not substantially homologous to the polypeptide. “Operatively linked” indicates that the polypeptide and the heterologous protein are fused in-frame. The heterologous protein can be fused to the N-terminus or C-terminus of the polypeptide. In one embodiment the fusion polypeptide does not affect function of the HMGB polypeptide per se. For example, the fusion polypeptide can be a GST-fusion polypeptide in which the polypeptide sequences are fused to the C-terminus of the GST sequences. Other types of fusion polypeptides include, but are not limited to, enzymatic fusion polypeptides, for example, β-galactosidase fusion polypeptides, yeast two-hybrid GAL fusion polypeptides, poly-His fusions, FLAG-tagged fusion polypeptides, GFP fusion polypeptides, and Ig fusion polypeptides. Such fusion polypeptides can facilitate the purification of recombinant polypeptide. In certain host cells (e.g., mammalian host cells), expression and/or secretion of a polypeptide can be increased by using a heterologous signal sequence. Therefore, in another embodiment, the fusion polypeptide contains a heterologous signal sequence at its N-terminus.

EP-A-O 464 533 discloses fusion proteins comprising various portions of immunoglobulin constant regions. The Fc is useful in therapy and diagnosis and thus results, for example, in improved pharmacokinetic properties (EP-A 0232 262). In drug discovery, for example, human proteins have been fused with Fc portions for the purpose of high-throughput screening assays to identify antagonists (Bennett et al., Journal of Molecular Recognition 8:52-58, 1995, and Johanson et al., J. Biol. Chem., 270(16):9459-9471, 1995). Thus, this invention also encompasses soluble fusion polypeptides containing a polypeptide of the invention and various portions of the constant regions of heavy or light chains of immunoglobulins of various subclass (IgG, IgM, IgA, IgE).

A chimeric or fusion polypeptide can be produced by standard recombinant DNA techniques. For example, DNA fragments coding for the different polypeptide sequences (e.g., an HMGB polypeptide and another polypeptide) are ligated together in-frame in accordance with conventional techniques. In another embodiment, the fusion gene can be synthesized by conventional techniques including automated DNA synthesizers. Alternatively, PCR amplification of nucleic acid fragments can be carried out using anchor primers that give rise to complementary overhangs between two consecutive nucleic acid fragments that can subsequently be annealed and re-amplified to generate a chimeric nucleic acid sequence (see Ausubel et al., Current Protocols in Molecular Biology, 1992). Moreover, many expression vectors are commercially available that already encode a fusion moiety (e.g., a GST moiety). A nucleic acid molecule encoding an HMGB polypeptide can be cloned into such an expression vector such that the fusion moiety is linked in-frame to the polypeptide.

HMGB functional equivalents can be generated using standard molecular biology techniques and assaying the function using, for example, methods described herein, such as, determining if the functional equivalent, when administered to a cell (e.g., a macrophage), increases release of a proinflammatory cytokine from the cell, as compared to an untreated control cell. In one embodiment, the HMGB functional equivalent has at least 50%, 60%, 70%, 80%, or 90% of the biological activity of the HMGB1 polypeptide of SEQ ID NO:1.

HMGB A Boxes

In particular embodiments, the compositions and methods of the present invention encompass HMGB A boxes. As used herein, an “HMGB A box”, also referred to herein as an “A box” (and also known as HMG A box), is a protein or polypeptide that has at least 50%, 60%, 70%, 75%, 80%, 85%, 90%, or 95%, sequence identity to an HMGB A box as described herein, and has one or more of the following biological activities: inhibiting inflammation mediated by HMGB and/or inhibiting release of a proinflammatory cytokine from a cell. In one embodiment, the HMGB A box polypeptide has one of the above biological activities. Typically, the HMGB A box polypeptide has both of the above biological activities. In one embodiment, the A box has at least 50%, 60%, 70%, 75%, 80%, 85%, 90%, or 95%, sequence identity to SEQ ID NO:4 and/or SEQ ID NO:7. In other embodiments, the HMGB A box has no more than 10%, 20%, 25%, 30%, 40%, 50%, 60%, 70%, 80%, or 90%, of the biological activity of full length HMGB. In another embodiment, the HMGB A box amino acid consists of the sequence of SEQ ID NO:4 or SEQ ID NO:7 (PTGKMSSYAF FVQTCREEHK KKHPDASVNF SEFSKKCSER WKTMSAKEKG KFEDMAKADK ARYEREMKTY IPPKGET) or the amino acid sequence in the corresponding region of an HMGB protein in a mammal. An HMGB A box is also a recombinantly-produced polypeptide having the same amino acid sequence as the A box sequences described above. The HMGB A box is preferably a vertebrate HMGB A box, for example, a mammalian HMGB A box, more preferably, a mammalian HMGB1 A box, for example, a human HMGB1 A box, and most preferably, the HMGB1 A box comprising or consisting of the sequence of SEQ ID NO:4 or SEQ ID NO:7.

An HMGB A box often has no more than about 85 amino acids and no fewer than about 4 amino acids. Examples of polypeptides having A box sequences within them include, but are not limited to, the HMGB polypeptides described herein. The A box sequences in such polypeptides can be determined and isolated using methods described herein, for example, by sequence comparisons to A boxes described herein and testing for biological activity using methods described herein and/or other method known in the art.

In addition to A boxes that can be found in the HMGB polypeptides described herein, other HMGB A box polypeptide sequences include the following sequences:

PDASVNFSEF SKKCSERWKT MSAKEKGKFE DMAKADKARY EREMKTYIPP KGET
(human HMGB1; SEQ ID NO:25);
DSSVNFAEF SKKCSERWKT MSAKEKSKFE DMAKSDKARY DREMKNYVPP KGDK
(human HMGB2; SEQ ID NO:26);
PEVPVNFAEF SKKCSERWKT VSGKEKSKFD EMAKADKVRY DREMKDYGPA KGGK
(human HMGB3; SEQ ID NO:27);
PDASVNFSEF SKKCSERWKT MSAKEKGKFE DMAKADKARY EREMKTYIPP KGET
(HMG1L5; SEQ ID NO:28);
SDASVNFSEF SNKCSERWKT MSAKEKGKFE DMAKADKTHY ERQMKTYIPP KGET
(HMG1L1; SEQ ID NO:29);
PDASVNFSEF SKKCSERWKA MSAKDKGKFE DMAKVDKADY EREMKTYIPP KGET
(HMG1L4; SEQ ID NO:30);
PDASVKFSEF LKKCSETWKT IFAKEKGKFE DMAKADKAHY EREMKTYIPP KGEK
(HMG sequence from BAC clone RP11-395A23; SEQ ID NO:31);
PDASINFSEF SQKCPETWKT TIAKEKGKFE DMAKADKAHY EREMKTYIPP KGET
(HMG1L9; SEQ ID NO:32);
PDASVNSSEF SKKCSERWKTMPTKQGKFE DMAKADRAH
(HMG1L8; SEQ ID NO:33);
PDASVNFSEF SKKCLVRGKT MSAKEKGQFE AMARADKARY EREMKTYIP PKGET
(LOC122441; SEQ ID NO:34);
LDASVSFSEF SNKCSERWKT MSVKEKGKFE DMAKADKACY EREMKIYPYL KGRQ
(LOC139603; SEQ ID NO:35);
and
GKGDPKKPRG KMSSYAFFVQ TCREEHKKKH PDASVNFSEF SKKCSERWKT MSAKEKGKFE
DMAKADKARY EREMKTYIPP KGET
(human HMGB1 A box; SEQ ID NO:36).

Functional equivalents of HMGB A boxes can also be used in the combination therapy compositions and methods of the present invention. In one embodiment, a functional equivalent of an HMGB A box inhibits release of a proinflammatory cytokine from a cell treated with an HMGB polypeptide. Examples of HMGB A box functional equivalents include, for example, biologically active fragments, post-translational modifications, variants, or fusion proteins comprising A boxes, as defined herein. A box functional equivalents can be generated using standard molecular biology techniques and assaying the function using known methods, for example, by determining if the fragment, when administered to a cell (e.g., a macrophage) decreases or inhibits release of a proinflammatory cytokine from the cell. In one embodiment, the A box functional equivalent has at least 50%, 60%, 70%, 80%, or 90% of the biological activity of the HMGB1 polypeptide of SEQ ID NO:4.

Optionally, the HMGB A box polypeptide is a substantially pure, or substantially pure and isolated, polypeptide that has been separated from components that naturally accompany it. The polypeptide may also be in an unpurified form, for example, in a cell, cell milieu, or cell extract. The critical feature is that the preparation allows for the desired function of the polypeptide, even in the presence of considerable amounts of other components.

HMGB B Boxes

In other embodiments, the compositions and methods of the present invention comprise antibodies to the HMGB B box or antigen-binding fragments thereof. As used herein, an “HMGB B box” also referred to herein as a “B box” (and also known as an HMG B box) is a polypeptide that has at least 60%, 65%, 70%, 75%, 80%, 85%, 90%, or 95%, sequence identity to SEQ ID NO:5 and/or SEQ ID NO:8 (as determined using the BLAST program and parameters described herein), lacks an A box, and has one or more of the following biological activities: increasing inflammation and/or increasing release of a proinflammatory cytokine from a cell. In one embodiment, the HMGB B box polypeptide has one of the above biological activities. Typically, the HMGB B box polypeptide has both of the above biological activities. Preferably, the HMGB B box has at least 25%, 30%, 40%, 50%, 60%, 70%, 80%, or 90%, of the biological activity of full length HMGB. In another embodiment, the HMGB box comprises or consists of the sequence of SEQ ID NO:5 or SEQ ID NO:8 (FKDPNAPKRL PSAFFLFCSE YRPKIKGEHP GLSIGDVAKK LGEMWNNTAA DDKQPYEKKA AKLKEKYEKD IAAY) or the amino acid sequence in the corresponding region of an HMGB protein in a mammal.

Preferably, the HMGB B box is a mammalian HMGB B box, for example, a human HMGB1 B box. An HMGB B box often has no more than about 85 amino acids and no fewer than about 4 amino acids. Examples of polypeptides having B box sequences within them include, but are not limited to, the HMGB polypeptides described herein. The B box sequences in such polypeptides can be determined and isolated using methods described herein, for example, by sequence comparisons to B boxes described herein and testing for biological activity using methods described herein and/or other method known in the art.

In addition to B boxes that can be found in the HMGB polypeptides described herein, other HMGB B box polypeptide sequences include the following sequences:

FKDPNAPKRP PSAFFLFCSE YRPKIKGEHP GLSIGDVAKK LGEMWNNTAA DDKQPYEKKA AKLKEKYEKD IAAY
(human HMGB1; SEQ ID NO:37);
KKDPNAPKRP PSAFFLFCSE HRPKIKSEHP GLSIGDTAKK LGEMWSEQSA KDKQPYEQKA AKLKEKYEKD IAAY
(human HMGB2; SEQ ID NO:38);
FKDPNAPKRL PSAFFLFCSE YRPKIKGEHP GLSIGDVAKK LGEMWNNTAA DDKQPYEKKA AKLKEKYEKD IAAY
(HMG1L5; SEQ ID NO:39);
FKDPNAPKRP PSAFFLFCSE YHPKIKGEHP GLSIGDVAKK LGEMWNNTAA DDKQPGEKKA AKLKEKYEKD IAAY
(HMG1L1; SEQ ID NO:40);
FKDSNAPKRP PSAFLLFCSE YCPKIKGEHP GLPISDVAKK LVEMWNNTFA DDKQLCEKKA AKLKEKYKKD TATY
(HMG1L4; SEQ ID NO:41);
FKDPNAPKRP PSAFFLFCSE YRPKIKGEHP GLSIGDVVKK LAGMWNNTAA ADKQFYEKKA AKLKEKYKKD IAAY
(HMG sequence from BAC clone RP11-359A23; SEQ ID NO:42);
and
FKDPNAPKRP PSAFFLFCSE YRPKIKGEHP GLSIGDVAKK LGEMWNNTAA DDKQPYEKKA AKLKEKYEKD
IAAYRAKGKP DAAKKGVVKA EK
(human HMGB1 box; SEQ ID NO:43).

Antibodies to functional equivalents of HMGB B boxes can also be used in the combination therapy compositions and methods of the present invention. Examples of HMGB B box functional equivalents include, for example, biologically active fragments, post-translational modifications, variants, or fusion proteins comprising B boxes, as defined herein. B box functional equivalents can be generated using standard molecular biology techniques and assaying the function using known methods, for example, by determining if the fragment, when administered to a cell (e.g., a macrophage) increases release of a proinflammatory cytokine from the cell. In one embodiment, the B box functional equivalent has at least 50%, 60%, 70%, 80%, or 90%, of the biological activity of the B box polypeptide of SEQ ID NO:5. Preferred examples of B box biological equivalents are polypeptides comprising, or consisting of, the first 20 amino acids of the B box (e.g., the first 20 amino acids of SEQ ID NO:5 (SEQ ID NO:44) or SEQ ID NO:8 (SEQ ID NO:45)).

Optionally, the HMGB B box polypeptide is a substantially pure, or substantially pure and isolated, polypeptide that has been separated from components that naturally accompany it. Alternatively, the polypeptide may be in an unpurified form, for example, in a cell, cell milieu, or cell extract. The critical feature is that the preparation allows for the desired function of the polypeptide, even in the presence of considerable amounts of other components.

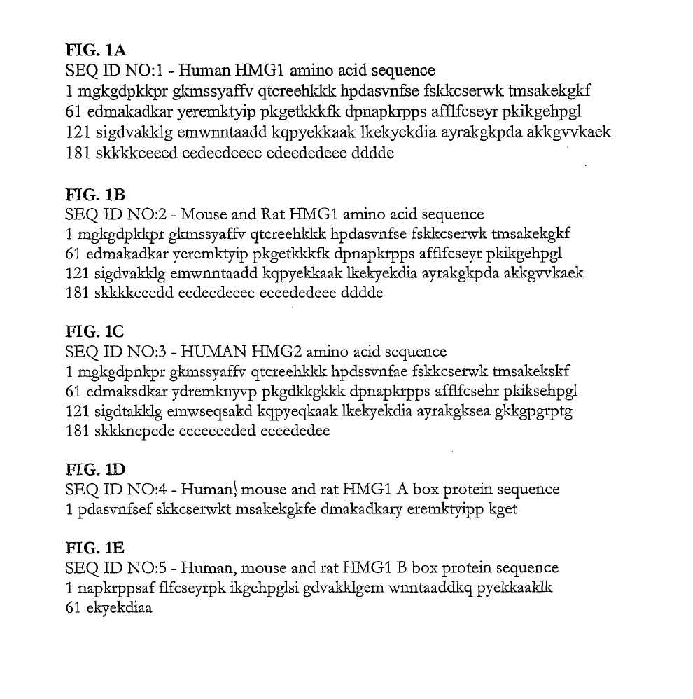

HMGB, HMGB A box, and/or HMGB B box functional equivalents, either naturally occurring or non-naturally occurring, include polypeptides that have sequence identity to the HMGB polypeptides, HMGB A boxes, and HMGB B boxes described herein. As used herein, two polypeptides (or regions of the polypeptides) are substantially homologous or identical when the amino acid sequences are at least about 60%, 70%, 75%, 80%, 85%, 90%, or 95%, or more homologous or identical. The percent identity of two amino acid sequences (or two nucleic acid sequences) can be determined by aligning the sequences for optimal comparison purposes (e.g., gaps can be introduced into one or both of the sequences). The amino acids or nucleotides at corresponding positions are then compared, and the percent identity between the two sequences is a function of the number of identical positions shared by the sequences (i.e., % identity=# of identical positions/total # of positions×100). In certain embodiments, the length of the HMGB polypeptide, HMGB A box polypeptide, or HMGB B box polypeptide aligned for comparison purposes is at least 30%, preferably, at least 40%, more preferably, at least 60%, and even more preferably, at least 70%, 80%, 90%, or 100%, of the length of the reference sequence, for example, those sequences provided in FIGS. 1A-1E, FIGS. 2A-2P, and SEQ ID NOs:25-43. The actual comparison of the two sequences can be accomplished by well-known methods, for example, using a mathematical algorithm. A preferred, non-limiting example of such a mathematical algorithm is described in Karlin et al. (Proc. Natl. Acad. Sci. USA, 90:5873-5877, 1993). Such an algorithm is incorporated into the BLASTN and BLASTX programs (version 2.2) as described in Schaffer et al. (Nucleic Acids Res., 29:2994-3005, 2001). When utilizing BLAST and Gapped BLAST programs, the default parameters of the respective programs (e.g., BLASTN; available at the Internet site for the National Center for Biotechnology Information) can be used. In one embodiment, the database searched is a non-redundant (NR) database, and parameters for sequence comparison can be set at: no filters; Expect value of 10; Word Size of 3; the Matrix is BLOSUM62; and Gap Costs have an Existence of 11 and an Extension of 1.

Another preferred, non-limiting example of a mathematical algorithm utilized for the comparison of sequences is the algorithm of Myers and Miller, CABIOS (1989). Such an algorithm is incorporated into the ALIGN program (version 2.0), which is part of the GCG (Accelrys, San Diego, Calif.) sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti (Comput. Appl. Biosci., 10:3-5, 1994); and FASTA described in Pearson and Lipman (Proc. Natl. Acad. Sci. USA, 85:2444-2448, 1988).

In another embodiment, the percent identity between two amino acid sequences can be accomplished using the GAP program in the GCG software package (Accelrys, San Diego, Calif.) using either a Blossom 63 matrix or a PAM250 matrix, and a gap weight of 12, 10, 8, 6, or 4 and a length weight of 2, 3, or 4. In yet another embodiment, the percent identity between two nucleic acid sequences can be accomplished using the GAP program in the GCG software package (Accelrys, San Diego, Calif.), using a gap weight of 50 and a length weight of 3.

As used herein, a “cytokine” is a soluble protein or peptide that is naturally produced by mammalian cells and that regulates immune responses and mediates cell-cell interactions. Cytokines can, either under normal or pathological conditions, modulate the functional activities of individual cells and tissues. A proinflammatory cytokine is a cytokine that is capable of causing one or more of the following physiological reactions associated with inflammation or inflammatory conditions: vasodilation, hyperemia, increased permeability of vessels with associated edema, accumulation of granulocytes and mononuclear phagocytes, and deposition of fibrin. In some cases, the proinflammatory cytokine can also cause apoptosis, such as in chronic heart failure, where TNF has been shown to stimulate cardiomyocyte apoptosis (Pulkki, Ann. Med. 29:339-343, 1997; and Tsutsui et al., Immunol. Rev. 174:192-209, 2000).

Nonlimiting examples of proinflammatory cytokines are tumor necrosis factor (TNF), interleukin (IL)-1α, IL-1β, IL-6, IL-8, IL-18, interferon γ, HMG-1, and macrophage migration inhibitory factor (MIF).

Proinflammatory cytokines are to be distinguished from anti-inflammatory cytokines, such as IL-4, IL-10, and IL-13, which are not mediators of inflammation.

In many instances, proinflammatory cytokines are produced in an inflammatory cytokine cascade, defined herein as an in vivo release of at least one proinflammatory cytokine in a mammal, wherein the cytokine release, directly or indirectly (e.g., through activation of, production of, or release of one or more cytokines or other molecules involved in inflammation from a cell), stimulates a physiological condition of the mammal. Thus, an inflammatory cytokine cascade is inhibited in embodiments of the invention where proinflammatory cytokine release causes a deleterious physiological condition.

Inhibition of release of a proinflammatory cytokine from a cell can be measured according to methods known to one skilled in the art. For example, TNF release from a cell can be measured using a standard murine fibroblast L929 (ATCC, American Type Culture Collection, Rockville, Md.) cytotoxicity bioassay (Bianchi et al., J. Exp. Med. 183:927-936, 1996) with the minimum detectable concentration of 30 pg/ml. The L929 cytotoxicity bioassay is carried out as follows. RAW 264.7 cells are cultured in RPMI 1640 medium (Life Technologies, Grand Island, N.Y.) supplemented with 10% fetal bovine serum (Gemini, Catabasas, Calif.), penicillin and streptomycin (Life Technologies). Polymyxin (Sigma, St. Louis, Mo.) is added at 100 units/ml to suppress the activity of any contaminating LPS. Cells are incubated with the combination therapy compositions described herein in Opti-MEM I medium for 8 hours, and conditioned supernatants (containing TNF which has been released from the cells) are collected. TNF which has been released from the cells is measured using a standard murine fibroblast L929 (ATCC) cytotoxicity bioassay (Bianchi et al., supra) with the minimum detectable concentration of 30 pg/ml. Recombinant mouse TNF is obtained from R & D Systems Inc. (Minneapolis, Minn.) and is used as a control in these experiments. Methods for measuring release of other cytokines from cells are known in the art.

Inflammatory cytokine cascades contribute to deleterious characteristics, including inflammatory conditions and cellular apoptosis. The composition and methods disclosed herein can be used to inhibit an inflammatory condition. In one embodiment, the inflammatory condition to be treated is one in which the inflammatory cytokine cascade causes a systemic reaction, such as endotoxic shock. In another embodiment, the inflammatory condition to be treated is one in which the inflammatory cytokine cascade is mediated by a localized inflammatory cytokine cascade, such as rheumatoid arthritis. In another embodiment, the inflammatory condition is selected from the group consisting of ileus, appendicitis, peptic, gastric or duodenal ulcers, inflammatory bowel disease, peritonitis, pancreatitis, ulcerative, pseudomembranous, acute or ischemic colitis, diverticulitis, epiglottitis, achalasia, cholangitis, cholecystitis, hepatitis, Crohn's disease, enteritis, Whipple's disease, asthma, allergy, anaphylactic shock, immune complex disease, organ ischemia, reperfusion ischemia, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, cystic fibrosis, pneumonitis, pneumoultramicroscopicsilicovolcanoconiosis, alvealitis, bronchiolitis, pharyngitis, pleurisy, sinusitis, influenza, respiratory syncytial virus infection, herpes infection, HIV infection, hepatitis B virus infection, hepatitis C virus infection, disseminated bacteremia, Dengue fever, candidiasis, malaria, filariasis, amebiasis, hydatid cysts, burns, dermatitis, dermatomyositis, sunburn, urticaria, warts, wheals, vasulitis, angiitis, endocarditis, arteritis, atherosclerosis, restenosis, thrombophlebitis, pericarditis, myocarditis, myocardial ischemia, periarteritis nodosa, rheumatic fever, Alzheimer's disease, coeliac disease, congestive heart failure, adult respiratory distress syndrome, meningitis, encephalitis, multiple sclerosis, cerebral infarction, cerebral embolism, Guillame-Barre syndrome, neuritis, neuralgia, spinal cord injury, paralysis, uveitis, arthritides, arthralgias, osteomyelitis, fasciitis, Paget's disease, gout, periodontal disease, arthritis, rheumatoid arthritis, synovitis, myasthenia gravis, thryoiditis, systemic lupus erythematosus, Goodpasture's syndrome, Behcets's syndrome, chronic obstructive pulmonary disease, psoriasis, allograft rejection, graft-versus-host disease, Type I diabetes, ankylosing spondylitis, Berger's disease, Retier's syndrome, and Hodgkins disease.

In other embodiments, the condition is selected from one or more of the group consisting of sepsis, peritonitis, pancreatitis, inflammatory bowel disease, ileus, ulcerative colitis, Crohn's disease, ischemia, for example, myocardial ischemia, organic ischemia, or reperfusion ischemia, cachexia, burns, adult respiratory distress syndrome, multiple sclerosis, atherosclerosis, restenosis, arthritis, rheumatoid arthritis, asthma, systemic lupus erythematosus, adult respiratory distress syndrome, chronic obstructive pulmonary disease, psoriasis, Behcet's syndrome, psoriasis, allograft rejection and graft-versus-host disease. Where the condition is allograft rejection, the composition may advantageously also include an immunosuppressant that is used to inhibit allograft rejection, such as cyclosporin.

When referring to the effect of any of the compositions or methods of the invention on the release of proinflammatory cytokines, the use of the terms “inhibit” or “decrease” encompasses at least a small but measurable reduction in proinflammatory cytokine release. In preferred embodiments, the release of the proinflammatory cytokine is inhibited by at least 10%, 20%, 25%, 30%, 40%, 50%, 75%, 80%, or 90% over non-treated controls. Inhibition can be assessed using methods described herein or other methods known in the art. Such reductions in proinflammatory cytokine release are capable of reducing the deleterious effects of an inflammatory cytokine cascade in in vivo embodiments.

Antibodies to HMGB, HMGB B Box and HMGB A Box Polypeptides

The present invention is directed in part to antibodies and antigen-binding fragments thereof that bind to an HMGB polypeptide or a biologically active fragment thereof (anti-HMGB antibodies). These antibodies and antigen-binding fragments can be combined with an agent that inhibits complement biological activity. The anti-HMGB antibodies and antigen-binding fragments can be neutralizing antibodies or antigen-binding fragments (i.e., can inhibit a biological activity of an HMG polypeptide or a fragment thereof, for example, the release of a proinflammatory cytokine from a vertebrate cell induced by HMGB). The invention also encompasses antibodies and antigen-binding fragments that selectively bind to an HMGB B box or a fragment thereof, but do not selectively bind to non-B box epitopes of HMGB (anti-HMGB B box antibodies and antigen-binding fragments thereof). The invention further encompasses antibodies and antigen-binding fragments that selectively bind to an HMGB A box or a functional equivalent thereof, but do not selectively bind to non-A box epitopes of HMGB (anti-HMGB A box antibodies and antigen-binding fragments thereof). In these embodiments, the antibodies and antigen-binding fragments can also be neutralizing antibodies and antigen-binding fragments (i.e., they can inhibit a biological activity of a HMGB polypeptide or a B box polypeptide or fragment thereof, for example, the release of a proinflammatory cytokine from a vertebrate cell induced by HMGB). Antibodies to HMGB have been shown to inhibit release of a proinflammatory cytokine from a cell treated with an HMGB polypeptide (see, for example, PCT publication WO 02/092004). Such antibodies can be combined with one or more agents that inhibit complement biological activity.

The term “antibody” or “purified antibody” as used herein refers to immunoglobulin molecules. The term “antigen-binding fragment” or “purified antigen-binding fragment” as used herein refers to immunologically active portions of immunoglobulin molecules, i.e., molecules that contain an antigen binding site that selectively bind to an antigen. A molecule that selectively binds to a polypeptide of the invention is a molecule that binds to that polypeptide or a fragment thereof, but does not substantially bind other molecules in a sample, e.g., a biological sample that naturally contains the polypeptide. Preferably the antibody is at least 60%, by weight, free from proteins and naturally occurring organic molecules with which it naturally associates. More preferably, the antibody preparation is at least 75% or 90%, and most preferably, 99%, by weight, antibody. Examples of immunologically active portions of immunoglobulin molecules include, but are not limited to Fv, Fab, Fab′ and F(ab′)₂ fragments. Such fragments can be produced by enzymatic cleavage or by recombinant techniques. For example, papain or pepsin cleavage can generate Fab or F(ab′)₂ fragments, respectively. Other proteases with the requisite substrate specificity can also be used to generate Fab or F(ab′)₂ fragments. Antibodies can also be produced in a variety of truncated forms using antibody genes in which one or more stop codons have been introduced upstream of the natural stop site. For example, a chimeric gene encoding a F(ab′)₂ heavy chain portion can be designed to include DNA sequences encoding the CH₁ domain and hinge region of the heavy chain.

The invention provides polyclonal and monoclonal antibodies that selectively bind to an HMGB B box polypeptide or an HMGB A box polypeptide of the invention. The term “monoclonal antibody” or “monoclonal antibody composition,” as used herein, refers to a population of antibody molecules that contain only one species of an antigen binding site capable of immunoreacting with a particular epitope of a polypeptide of the invention. A monoclonal antibody composition thus typically displays a single binding affinity for a particular polypeptide of the invention with which it immunoreacts.

Polyclonal antibodies can be prepared as described herein by immunizing a suitable subject with a desired immunogen, e.g., an HMGB polypeptide, an HMGB B box polypeptide, an HMGB A box polypeptide or fragments thereof. The antibody titer in the immunized subject can be monitored over time by standard techniques, such as with an enzyme linked immunosorbent assay (ELISA) using immobilized polypeptide. If desired, the antibody molecules directed against the polypeptide can be isolated from the mammal (e.g., from the blood) and further purified by well-known techniques, such as protein A chromatography to obtain the IgG fraction.

At an appropriate time after immunization, e.g., when the antibody titers are highest, antibody-producing cells can be obtained from the subject and used to prepare monoclonal antibodies by standard techniques, such as the hybridoma technique originally described by Kohler and Milstein (Nature 256:495-497, 1975), the human B cell hybridoma technique (Kozbor et al., Immunol. Today 4:72, 1983), the EBV-hybridoma technique (Cole et al., Monoclonal Antibodies and Cancer Therapy, Alan R. Liss, Inc., pp. 77-96, 1985) or trioma techniques. The technology for producing hybridomas is well known (see generally Current Protocols in Immunology, Coligan et al., (eds.) John Wiley & Sons, Inc., New York, N.Y., 1994). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds a polypeptide described herein.

Any of the many well known protocols used for fusing lymphocytes and immortalized cell lines can be applied for the purpose of generating a monoclonal antibody to a polypeptide of the invention (see, e.g., Current Protocols in Immunology, supra; Galfre et al., Nature, 266:55052, 1977; R. H. Kenneth, in Monoclonal Antibodies: A New Dimension In Biological Analyses, Plenum Publishing Corp., New York, N.Y., 1980; and Lerner, Yale J. Biol. Med. 54:387-402, 1981). Moreover, the ordinarily skilled worker will appreciate that there are many variations of such methods that also would be useful.

In one alternative to preparing monoclonal antibody-secreting hybridomas, a monoclonal antibody to an HMGB polypeptide, an HMGB B box polypeptide or an HMGB A box polypeptide of the invention can be identified and isolated by screening a recombinant combinatorial immunoglobulin library (e.g., an antibody phage display library) with the polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display libraries can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., Bio/Technology 9:1370-1372, 1991; Hay et al., Hum. Antibod. Hybridomas 3:81-85, 1992; Huse et al., Science 246:1275-1281, 1989; and Griffiths et al., EMBO J. 12:725-734, 1993.

Single chain antibodies, and recombinant antibodies, such as chimeric, humanized, primatized (CDR-grafted) or veneered antibodies, as well as chimeric, CDR-grafted or veneered single chain antibodies, comprising portions derived from different species, and the like are also encompassed by the present invention and the term “antibody”. The various portions of these antibodies can be joined together chemically by conventional techniques, or can be prepared as a contiguous protein using genetic engineering techniques. For example, nucleic acids encoding a chimeric or humanized chain can be expressed to produce a contiguous protein. See, e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al, European Patent No. 0 451 216 B1; and Padlan, E. A. et al., EP 0 519 596 A1. See also, Newman, R. et al., BioTechnology, 10: 1455-1460 (1992), regarding primatized antibody, and Ladner et al., U.S. Pat. No. 4,946,778 and Bird, R. E. et al., Science, 242: 423-426 (1988)) regarding single chain antibodies. Techniques for the production of single chain antibodies (U.S. Pat. No. 4,946,778) can be adapted to produce single chain antibodies to the HMGB polypeptides or HMGB B box polypeptides or fragments thereof. Also, transgenic mice, or other organisms such as other mammals, may be used to express humanized antibodies.

Humanized antibodies can be produced using synthetic or recombinant DNA technology using standard methods or other suitable techniques. Nucleic acid (e.g., cDNA) sequences coding for humanized variable regions can also be constructed using PCR mutagenesis methods to alter DNA sequences encoding a human or humanized chain, such as a DNA template from a previously humanized variable region (see e.g., Kamman, M., et al., Nucl. Acids Res., 17: 5404 (1989)); Sato, K., et al., Cancer Research, 53: 851-856 (1993); Daugherty, B. L. et al., Nucleic Acids Res., 19(9): 2471-2476 (1991); and Lewis, A. P. and J. S. Crowe, Gene, 101: 297-302 (1991)). Using these or other suitable methods, variants can also be readily produced. In one embodiment, cloned variable regions can be mutated, and sequences encoding variants with the desired specificity can be selected (e.g., from a phage library; see e.g., Krebber et al., U.S. Pat. No. 5,514,548; Hoogenboom et al., WO 93/06213).

If the antibody is used therapeutically in in vivo applications, the antibody can be modified to make it less immunogenic. For example, if the individual is human the antibody is preferably “humanized”; where the complementarity determining region(s) (CDRs) of the antibody is transplanted into a human antibody (for example, as described in Jones et al., Nature 321:522-525, 1986; and Tempest et al., Biotechnology 9:266-273, 1991). The antibody can be a humanized antibody comprising one or more immunoglobulin chains, said antibody comprising a CDR of nonhuman origin (e.g., one or more CDRs derived from an antibody of nonhuman origin) and a framework region derived from a light and/or heavy chain of human origin (e.g., CDR-grafted antibodies with or without framework changes). In one embodiment, the antibody or antigen-binding fragment thereof comprises the light chain CDRs (CDR1, CDR2 and CDR3) and heavy chain CDRs (CDR1, CDR2 and CDR3) of a particular immunoglobulin. In another embodiment, the antibody or antigen-binding fragment further comprises a human framework region.

Human antibodies and nucleic acids encoding the same can be obtained from a human or from human-antibody transgenic animals. Human-antibody transgenic animals (e.g., mice) are animals that are capable of producing a repertoire of human antibodies, such as XENOMOUSE (Abgenix, Fremont, Calif.), HUMAB-MOUSE, KIRIN TC MOUSE or KM-MOUSE (MEDAREX, Princeton, N.J.). Generally, the genome of human-antibody transgenic animals has been altered to include a transgene comprising DNA from a human immunoglobulin locus that can undergo functional rearrangement. An endogenous immunoglobulin locus in a human-antibody transgenic animal can be disrupted or deleted to eliminate the capacity of the animal to produce antibodies encoded by an endogenous gene. Suitable methods for producing human-antibody transgenic animals are well known in the art. (See, for example, U.S. Pat. Nos. 5,939,598 and 6,075,181 (Kucherlapati et al.), U.S. Pat. Nos. 5,569,825, 5,545,806, 5,625,126, 5,633,425, 5,661,016, and 5,789,650 (Lonberg et al.), Jakobovits et al., Proc. Natl. Acad. Sci. USA, 90: 2551-2555 (1993), Jakobovits et al., Nature, 362: 255-258 (1993), Jakobovits et al. WO 98/50433, Jakobovits et al. WO 98/24893, Lonberg et al. WO 98/24884, Lonberg et al. WO 97/13852, Lonberg et al. WO 94/25585, Lonberg et al. EP 0 814 259 A2, Lonberg et al. GB 2 272 440 A, Lonberg et al., Nature 368:856-859 (1994), Lonberg et al., Int Rev Immunol 13(1):65-93 (1995), Kucherlapati et al. WO 96/34096, Kucherlapati et al. EP 0 463 151 B1, Kucherlapati et al. EP 0 710 719 A1, Surani et al. U.S. Pat. No. 5,545,807, Bruggemann et al. WO 90/04036, Bruggemann et al. EP 0 438 474 B1, Taylor et al., Int. Immunol. 6(4)579-591 (1994), Taylor et al., Nucleic Acids Research 20(23):6287-6295 (1992), Green et al., Nature Genetics 7:13-21 (1994), Mendez et al., Nature Genetics 15:146-156 (1997), Tuaillon et al., Proc Natl Acad Sci USA 90(8):3720-3724 (1993) and Fishwild et al., Nat Biotechnol 14(7):845-851 (1996), the teachings of each of the foregoing are incorporated herein by reference in their entirety.)

Because vertebrate HMGB polypeptides, HMGB B boxes and HMGB A boxes show a high degree of sequence conservation, it is reasonable to believe that antibodies that bind to vertebrate HMGB polypeptides, HMGB B boxes or HMGB A boxes in general can induce release of a proinflammatory cytokine from a vertebrate cell. Therefore, antibodies against vertebrate HMGB polypeptides or HMGB B boxes without limitation are within the scope of the invention. In one embodiment, the antibodies are neutralizing antibodies.

Phage display technology can also be utilized to select antibody genes with binding activities towards the polypeptide either from repertoires of PCR amplified v-genes of lymphocytes from humans screened for possessing anti-B box antibodies or from naive libraries (McCafferty et al., Nature 348:552-554, 1990; and Marks, et al., Biotechnology 10:779-783, 1992). The affinity of these antibodies can also be improved by chain shuffling (Clackson et al., Nature 352: 624-628, 1991).

When the antibodies are obtained that specifically bind to HMGB epitopes, HMGB B box epitopes or HMGB A box epitopes, they can then be screened without undue experimentation for the ability to inhibit release of a proinflammatory cytokine using standard methods. Anti-HMGB antibodies, anti-HMGB B box antibodies and anti-HMGB A box antibodies that can inhibit the production of any single proinflammatory cytokine, and/or inhibit the release of a proinflammatory cytokine from a cell, and/or inhibit the a condition characterized by activation of an inflammatory cytokine cascade are within the scope of the present invention. Preferably, the antibodies can inhibit the production of TNF, IL-1, or IL-6.

Polyclonal antibodies raised against HMGB have been produced (see, for example, U.S. Pat. No. 6,468,555 B1, the entire teachings of which are incorporated herein by reference). These antibodies have been shown to inhibit release of a proinflammatory cytokine from a cell, and to treat inflammation.

Polyclonal antibodies against the HMGB1 B box have been raised in rabbits (Cocalico Biologicals, Inc., Reamstown, Pa.) and assayed for titer by immunoblotting. IgG was purified from anti-HMGB1 antiserum using Protein A agarose according to manufacturer's instructions (Pierce, Rockford, Ill.) (see, for example, PCT Publication No. WO 02/092004). Anti-HMGB1 B box antibodies were affinity purified using cyanogen bromide activated Sepharose beads (Cocalico Biological, Inc.). Non-immune rabbit IgG was purchased from Sigma (St. Louis, Mo.). Antibodies detected full length HMGB1 and HMGB1 B box in immunoassays, but did not cross react with TNF, IL-1 or IL-6. These HMGB1 B box antibodies also inhibited release of a proinflammatory cytokine from a cell and provided protection against sepsis induced by cecal ligation and puncture.

Monoclonal antibodies to HMGB1 are known in the art, and are taught, for example, in WO 2005/026209 and U.S. Provisional Application No. 60/502,568, entitled “Monoclonal Antibodies Against HMGB1”, by Walter Newman, Shixin Qin, Theresa O'Keefe and Robert Obar, filed on Sep. 11, 2003, Attorney Docket No. 3258.1033-000; the entire teachings of which are incorporated herein by reference. Particular monoclonal antibodies to HMGB1 include, e.g., 6E6 HMGB1 mAb, 2E11 HMGB1 mAb, 6H9 HMGB1 mAb, 10D4 HMGB1 mAb and 2G7 HMGB1 mAb.

6E6 HMGB1 mAb, also referred to as 6E6-7-1-1 or 6E6, can be produced by murine hybridoma 6E6 HMGB1 mAb, which was deposited on Sep. 3, 2003, on behalf of Critical Therapeutics, Inc., 675 Massachusetts Avenue, 14^{th} Floor, Cambridge, Mass. 02139, U.S.A., at the American Type Culture Collection, 10801 University Boulevard, Manassas, Va. 20110, U.S.A., under Accession No. PTA-5433.

2E11 HMGB1 mAb, also referred to as 2E11-1-1-2 or 2E11, can be produced by murine hybridoma 2E11 HMGB1 mAb, which was deposited on Sep. 3, 2003, on behalf of Critical Therapeutics, Inc., 675 Massachusetts Avenue, 14^{th} Floor, Cambridge, Mass. 02139, U.S.A., at the American Type Culture Collection, 10801 University Boulevard, Manassas, Va. 20110, U.S.A., under Accession No. PTA-5431.

6H9 HMGB1 mAb, also referred to as 6H9-1-1-2 or 6H9, can be produced by murine hybridoma 6H9 HMGB1 mAb, which was deposited on Sep. 3, 2003, on behalf of Critical Therapeutics, Inc., 675 Massachusetts Avenue, 14^{th} Floor, Cambridge, Mass. 02139, U.S.A., at the American Type Culture Collection, 10801 University Boulevard, Manassas, Va. 20110, U.S.A., under Accession No. PTA-5434.

10D4 HMGB1 mAb, also referred to as 10D4-1-1-1-2 or 10D4, can be produced by murine hybridoma 10D4 HMGB1 mAb, which was deposited on Sep. 3, 2003, on behalf of Critical Therapeutics, Inc., 675 Massachusetts Avenue, 14^{th} Floor, Cambridge, Mass. 02139, U.S.A., at the American Type Culture Collection, 10801 University Boulevard, Manassas, Va. 20110, U.S.A., under Accession No. PTA-5435.

2G7 HMGB1 mAb, also referred to as 3-2G7-1-1-1 or 2G7, can be produced by murine hybridoma 2G7 HMGB1 mAb, which was deposited on Sep. 3, 2003, on behalf of Critical Therapeutics, Inc., 675 Massachusetts Avenue, 14^{th} Floor, Cambridge, Mass. 02139, U.S.A., at the American Type Culture Collection, 10801 University Boulevard, Manassas, Va. 20110, U.S.A., under Accession No. PTA-5432.

As described herein, in certain embodiments the compositions and methods of the invention comprise or utilize antibodies or antigen-binding fragments thereof, that bind an HMGB polypeptide or fragment thereof (e.g., an HMGB B box or a biologically active fragment thereof, an HMGB A box or a biologically active fragment thereof). Such HMGB polypeptides include, e.g., those HMGB polypeptides described herein. In one embodiment, the antibody or antigen-binding fragment binds a mammalian HMGB polypeptide. In another embodiment, the antibody or antigen-binding fragment binds an HMGB1 polypeptide. In yet another embodiment, the antibody or antigen-binding fragment binds an HMGB1 polypeptide consisting of SEQ ID NO:1.

In one embodiment, the antibody or antigen-binding fragment binds an HMGB B box or a biologically active fragment thereof. In another embodiment, the antibody or antigen-binding fragment binds an HMGB B box consisting of SEQ ID NO:5. In yet another embodiment, the antibody or antigen-binding fragment binds a biologically active fragment of an HMGB B Box consisting of SEQ ID NO:45.

In one embodiment, the antibody or antigen-binding fragment binds an HMGB A box or a biologically active fragment thereof. In another embodiment, the antibody or antigen-binding fragment binds an HMGB A box consisting of SEQ ID NO:4. In yet another embodiment, the antibody or antigen-binding fragment binds a biologically active fragment of an HMGB A Box.

Inhibitors of HMGB Receptor Binding and/or HMGB Signaling

In particular embodiments, the invention is directed to combination therapy compositions comprising an inhibitor of HMGB receptor binding and/or signaling and an agent that inhibits complement biological activity. Such combination therapy compositions can be used for the treatment of inflammatory conditions, as described herein.

Inhibitors of HMGB receptor binding and/or signaling include, e.g., polypeptides comprising a high mobility group box (HMGB) A box (as described herein), antibodies to HMGB and/or HMGB B boxes and antigen-binding fragments thereof (as described herein), HMGB small molecule antagonists (e.g., ethyl pyruvate, certain derivatives of isoxazole, isoxazolidine, isothiazole and isothiazolidine compounds), antibodies to TLR2, soluble TLR2, TLR2 small molecule antagonists, TLR2 dominant mutant proteins, antibodies to TLR4, soluble TLR4, TLR4 small molecule antagonists, TLR4 dominant mutant proteins, antibodies to RAGE, soluble RAGE, RAGE small molecule antagonists (e.g., as taught in PCT Publication Nos. WO 01/99210, WO 02/069965 and WO 03/075921 and U.S. Published Application No. US 2002/0193432A1), and RAGE dominant mutant proteins. Inhibitors of HMGB receptor binding and/or signaling also include, e.g., antisense and small double-stranded interfering RNA (RNA interference (RNAi) that target HMGB, TLR2, TLR4 and/or RAGE proteins.

In one embodiment, the inhibitors of HMGB receptor binding and/or signaling is an HMGB small molecule antagonist. As used herein, an HMGB small molecule antagonist is a molecule that antagonizes production of HMGB and/or one or more biological activities of HMGB (e.g., HMGB-mediated signaling, HMGB-mediated increase in inflammation, HMGB-mediated increase in release of a proinflammatory cytokine from a cell). Such HMGB small molecule antagonists include those small molecule antagonists that bind directly to HMGB, thereby inhibiting HMGB receptor binding and/or signaling, as well as those small molecule antagonists that do not bind to HMGB but antagonize production of HMGB and/or one or more biological activities of HMGB (e.g., HMGB-mediated signaling, HMGB-mediated increase in inflammation, HMGB-mediated increase in release of a proinflammatory cytokine from a cell). HMGB small molecule antagonists typically have a molecular weight of 1000 or less, 500 or less, 250 or less or 100 or less. Suitable HMGB small molecule antagonists include but are not limited to, an ester of an alpha-ketoalkanoic acid including, for example, ethyl pyruvate (see, e.g., PCT Publication WO 02/074301; the entire teachings of which are incorporated herein by reference) and certain derivatives of isoxazole, isoxazolidine, isothiazole and isothiazolidine compounds (e.g., as taught in U.S. Application No. 60/516,027, entitled “Anti-Inflammatory Compounds”, filed Oct. 31, 2003, Attorney Docket No. 3268.1007-001; the entire teachings of which are incorporated herein by reference).

For example, it has been shown that an ester of an alpha-ketoalkanoic acid can inhibit the release of proinflammatory cytokines such as TNF, IL-1β and HMGB1. See, e.g., PCT Publication WO 02/074301, the entire teachings of which are incorporated herein by reference. Therefore, in one embodiment of the invention, the HMGB small molecule antagonist is an ester of an alpha-ketoalkanoic acid. In another embodiment, the HMGB small molecule antagonist is an ester of a C3 to C8, straight chained or branched alpha-ketoalkanoic acid. In an additional embodiment, the HMGB small molecule antagonist is selected from the group consisting of alpha-keto-butyrate, alpha-ketopentanoate, alpha-keto-3-methyl-butyrate, alpha-keto-4-methyl-pentanoate or alpha-keto-hexanoate. A variety of groups are suitable for the ester portion of the molecule, e.g., alkyl, aralkyl, alkoxyl, carboxyalkyl, glyceryl or dihydroxyacetone. Specific examples include ethyl, propyl, butyl, carboxymethyl, acetoxymethyl, carbethoxymethyl and ethoxymethyl. Ethyl esters are preferred. In a further embodiment, the HMGB small molecule antagonist is an ethyl, propyl, butyl, carboxymethyl, acetoxymethyl, carbethoxymethyl and ethoxymethyl ester. In an additional preferred embodiment, the HMGB small molecule antagonist is an ester of pyruvic acid. In a further preferred embodiment, the HMGB small molecule antagonist is ethyl pyruvate. Thiolesters (e.g., wherein the thiol portion is cysteine or homocysteine) are also included.

In another preferred embodiment, the HMGB small molecule antagonist is selected from the group consisting of ethyl pyruvate, propyl pyruvate, carboxymethyl pyruvate, acetoxymethyl pyruvate, carbethoxymethyl pyruvate, ethoxymethyl pyruvate, ethyl alpha-keto-butyrate, ethyl alpha-keto-pentanoate, ethyl alpha-keto-4-methyl-pentanoate and ethyl-keto-hexanoate. In an additional preferred embodiment, the HMGB small molecule antagonist is ethyl pyruvate.

It has been shown that certain derivatives of isoxazole, isoxazolidine, isothiazole and isothiazolidine compounds are HMGB small molecule antagonists that inhibit production and release of certain proinflammatory cytokines (e.g., TNF, HMGB1). See, e.g., U.S. Application No. 60/516,027, filed Oct. 31, 2003, Attorney Docket No. 3268.1007-001, the entire teachings of which are incorporated herein by reference. Such derivatives include, for example, a compound of Formula (I) or a pharmaceutically acceptable salt thereof:

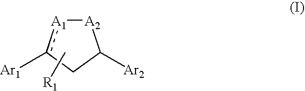

wherein Ar₁ and Ar₂ are independently a monocyclic six-member optionally substituted heteroaryl group;

A₁ is ═N— or —NR^{a}— and A₂ is O or S; R^{a} is H or C1-C6 alkyl;

R₁ is selected from —H, C1-C6 alkyl, phenyl, C1-C6 haloalkyl, halogen, —OH, —OR^{b}, C1-C6 hydroxyalkyl, C1-C6 alkoxyalkyl, —O(C1-C6 haloalkyl), —SH, —SR^{b}, —NO₂, —CN, —NR^{b}CO₂R^{b}, —NR^{b}C(O)R^{b}, —CO₂R^{b}, —C(O)R^{b}, —C(O)N(R^{b})₂, —OC(O)R^{b} and —NR^{b}R^{b}.

Each R^{b} is H or a C1-C6 alkyl group.

In one embodiment, the HMGB small molecule antagonist is a compound represented by Formula (I a) or a pharmaceutically acceptable salt thereof:

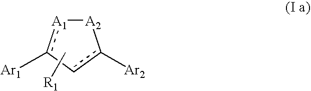

wherein Ar₁, Ar₂, A₁, A₂, R₁ and its substituents are defined above for Formula (I).

In another embodiment, the HMGB small molecule antagonist is a compound represented by Formula (VII) or a pharmaceutically acceptable salt thereof:

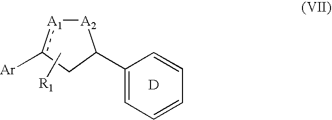

wherein Ar is an optionally substituted, monocyclic, six-member heteroaryl;

A₁ is ═N— or —NR^{a}— and A₂ is O or S;

R₁ is —H, C1-C6 alkyl, phenyl, C1-C6 haloalkyl, halogen, —OH, —OR^{b}, C1-C6 hydroxyalkyl, C1-C6 alkoxyalkyl, —O(C1-C6 haloalkyl), —SH, —SR^{b}, —NO₂, —CN, —NR^{b}CO₂R^{b}, —NR^{b}C(O)R^{b}, —CO₂R^{b}, —C(O)R^{b}, —C(O)N(R^{b})₂, —OC(O)R^{b} or —NR^{b}R^{b};

each Rₐ is —H or C1-C6 alkyl and each R^{b} is —H or a C1-C6 alkyl group;

ring D is optionally substituted with zero, one or more substituents other than amide and is not an alkylphenol.

In another embodiment, the HMGB small molecule antagonist is a compound represented by Formula (VII a) or a pharmaceutically acceptable salt thereof:

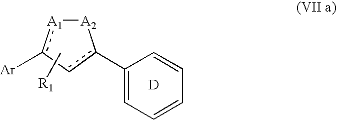

wherein Ar, A₁, A₂, R₁ and ring D and its substituents are as defined above for Formula (VII).

In another embodiment, the HMGB small molecule antagonist is a compound represented by Formulae (II), (III) or (IV):

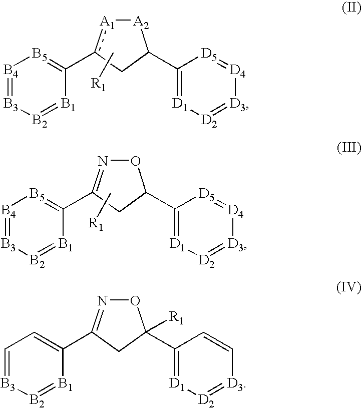

The variables for Formulae (II) to (IV) are described below.

B₁ through B₅ and D₁ through D₅ are independently N or CR^{c}, provided that from one to three of B₁ through B₅ and from one to three of D₁ through D₅ are N. Each R^{c} is independently any suitable substituent as described below for a heteroaryl group. R₁ is as described above for Formula (I).

More preferably, in structural Formulae (II) to (IV), R₁ is —H or a C1-C3 alkyl, optionally substituted with a halogen or a hydroxyl, and/or R^{c} is —H, halogen, —NO₂, —CN, C1-C3 alkyl, C1-C3 haloalkyl, C1-C3 hydroxyalkyl, C1-C3 alkoxyalkyl, —N(R^{d})₂, —NR^{d}C(O)R^{d}, or —C(O)N(R^{d})₂. R^{d} is H or C1-C3 alkyl.

In another embodiment, the HMGB small molecule antagonist is a compound represented by Formulae (V a) through (V i):

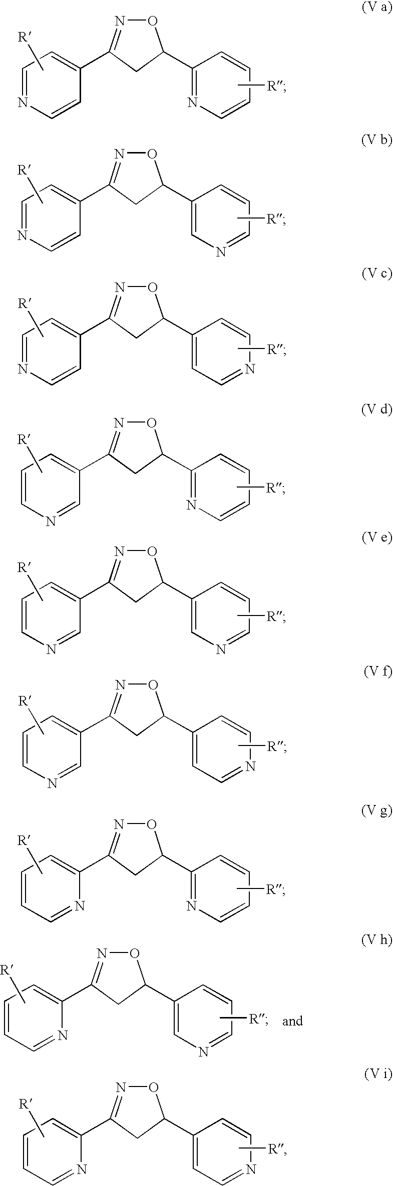

wherein R′ and R″ are independently —H, halogen, —NO₂, —CN, C1-C3 alkyl, C1-C3 haloalkyl, C1-C3 hydroxyalkyl, C1-C3 alkoxyalkyl, —N(R^{d})₂, —NR^{d}C(O)R^{d}, or —C(O)N(R^{d})₂. R^{d} is as defined above.

Other specific examples of HMGB small molecule antagonists include:

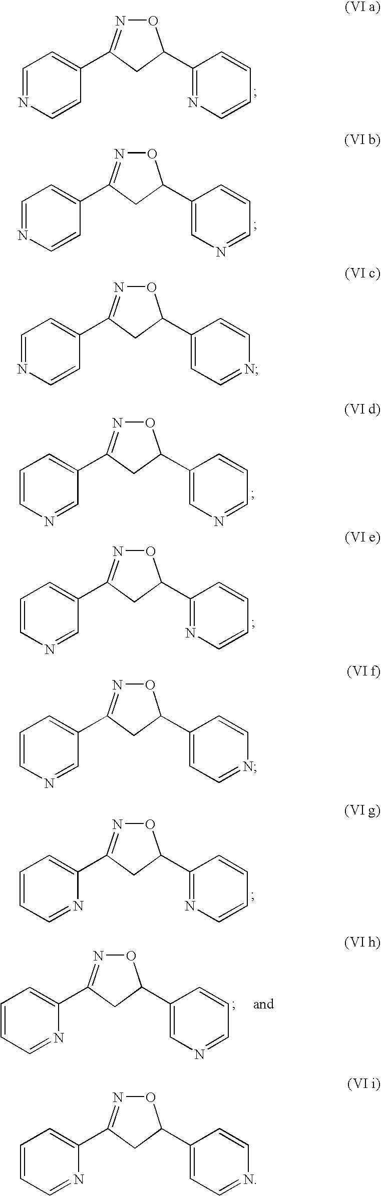

In one embodiment, the HMGB small molecule antagonists are represented by Formula (VI d) or (VI f).

In another embodiment, the HMGB small molecule antagonist is a compound represented by Formulae (I), (I a), (II), (III), (IV), (V a) through (V i) and (VI a) through (V i) or pharmaceutically acceptable salts thereof.

In one embodiment, the HMGB small molecule antagonist is represented by Formula (II a):

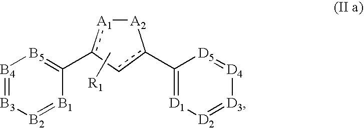

wherein variables B₁ through B₅, D₁ through D₅, A₁, A₂ and R₁ are defined above for Formula (I).

In another embodiment, the HMGB small molecule antagonist is represented by Formula (VII). The variables of Formula (VII) are as described above. In still other embodiments, the compounds of Formula (VII) are represented by Formulae (VIII) and (IX):

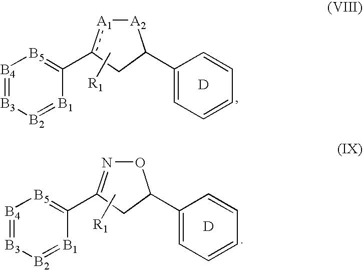

The variables of Formulae (VIII) and (IX) are as follows.

A₁, A₂ and R₁ are as defined above for Formula (VII);

B₁ through B₅ are independently N or CR^{c}, provided that from one to three of B₁ through B₅₁ are N;

Each R^{c} is independently any suitable substituent as described below for a heteroaryl group;

Ring D is optionally substituted with zero, one or more substituent R₂. Each R₂ is independently any suitable substituent described below for an aryl group;

In yet another embodiment, the HMGB small molecule antagonist is represented by Formulae (X) and (XI a) to (XI c):

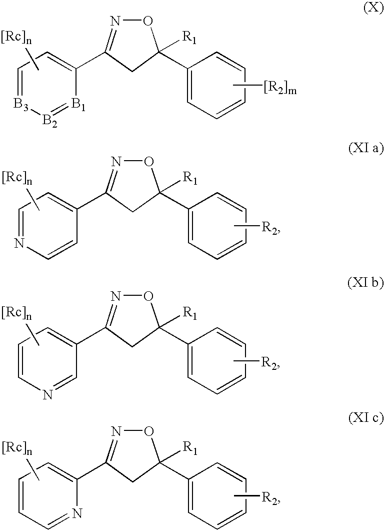

wherein one of B₁ through B₃ is N, n is 0, 1 or 2 and m is 0, 1 or 2.

Preferably, in Formulae (VIII) to (XI a) through (XI c) R^{c} is —H, halogen, —NO₂, —CN, C1-C3 alkyl, C1-C3 haloalkyl, C1-C3 hydroxyalkyl, C1-C3 alkoxyalkyl, —N(R^{d})₂, —NR^{d}C(O)R^{d}, or —C(O)N(R^{d})₂ and R₂ is —H, halogen, —NO₂, —CN, C1-C3 alkyl, C1-C3 haloalkyl, C1-C3 hydroxyalkyl or C1-C3 alkoxyalkyl. R^{d} is H or C1-C3 alkyl.

Specific examples of HMGB small molecule antagonists suitable for using in pharmaceutical compositions of the present invention are:

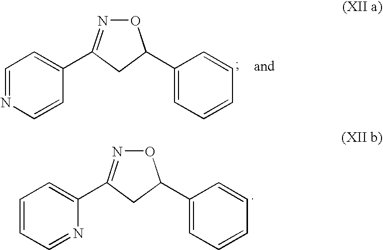

In one embodiment, the HMGB small molecule antagonist represented by Formula (VII a), is further represented by Formula (VIII a):

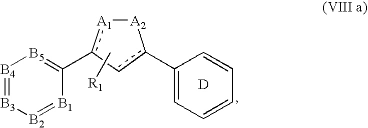

wherein B₁ through B₅ and the substituents thereof are defined above for Formula (VIII).

In another embodiment, the HMGB small molecule antagonist is represented by any one of Formulae (I), (I a), (II), (II a) (III), (IV), (V a) to (V i), (VI a) to (VI i), (VII), (VII a), (Vi), (IX), (X), (XI a) through (XI c), (XII a) and (XII b) as defined above.

The term “heteroaryl”, as used herein, refers to aromatic groups containing one or more heteroatoms (O, S, or N). A heteroaryl group of the present invention is a monocyclic six-member group. The heteroaryl groups of this invention can also include ring systems substituted with one or more oxo moieties. Examples of heteroaryl groups include, but are not limited to, pyridinyl, pyridazinyl, pyrimidinyl, pyrazinyl, pyridazinyl, pyridazinyl and triazinyl.

Suitable substituents on a heteroaryl group (including heteroaryl groups represented by Ar, Ar₁ and Ar₂) are those that do not substantially interfere with the pharmaceutical activity of the disclosed compound. A heteroaryl may have one or more substituents, which can be identical or different. Examples of suitable substituents for a substitutable carbon atom in a heteroaryl group include —H, C1-C6 alkyl, halogen, C1-C6 haloalkyl, —R^{o}, —OH, —OR^{o}, —O(C1-C6 haloalkyl), —SH, —SR^{o}, —NO₂, —CN, —NH₂, —NHCO₂R^{o}, —NHC(O)H, —NHC(O)R^{o}, —NR^{o}C(O)R^{o}, —CO₂H, —CO₂R^{o}, —C(O)H, —C(O)R^{o}, —C(O)NHR^{o}, —C(O)NR^{o})₂, —OC(O)R^{o}, —S(O)₂R^{o}, —SO₂NH₂, —S(O)R^{o}, —NHSO₂R^{o}, or a C1-C6 alkyl group substituted with R^{o}, —OH, —OR^{o}, —SH, —SR^{o}, —NO₂—CN, —NHCO₂R^{o}, —NHC(O)H, —NHC(O)R^{o}, —CO₂H, —CO₂R^{o}, —C(O)H, —C(O)R^{o}, —C(O)NHR^{o}, —OC(O)R^{o}, —S(O)₂R^{o}, —SO₂NH₂, —S(O)R^{o} or —NHSO₂R^{o}. R^{o} is independently, C1-C6 alkyl, aryl or heteroaryl group and wherein the aryl or heteroaryl group represented by R^{o} is optionally substituted with one or more halogen, methyl or methoxy groups.

The term “aryl”, as used herein, refers to a carbocyclic aromatic group. Examples of aryl groups include, but are not limited to phenyl and naphthyl.

A substituted aryl group can have one or more substituents which can be the same or different. Suitable substituents for a substituted aryl group, including ring D, typically represented herein as “R₂”, are as defined above for a heteroaryls, provided that the substituents on ring D are other than amide and that ring D is not an alkylphenol. As used herein, the term “amide” refers to a —C(O)NHR^{o} group, where R^{o} is defined above for heteroaryl groups. As used herein, the term “alkylphenol” refers to a six-member monocyclic aryl substituted with one hydroxyl groups and one or more alkyls. Examples of suitable substituents for an aryl include —H, halogen, C1-C6 haloalkyl, —R^{o}, —OH, —OR^{o}, —O(C1-C6 haloalkyl), —SH, —SR^{o}, —NO₂, —CN, —NHCO₂R^{o}, —NHC(O)H, —NHC(O)R^{o}, —CO₂H, —CO₂R^{o}, —C(O)H, —C(O)R^{o}, —OC(O)R^{o}, —S(O)₂R^{o}, —SO₂NH₂, —S(O)R^{o}, —NHSO₂R^{o}, or a C1-C6 alkyl group substituted with R^{o}, —OH, —OR^{o}, —SH, —SR^{o1}, —NO₂, —CN, —NHCO₂R^{o}, —NHC(O)H, —NHC(O)R^{o}, —CO₂H, —CO₂R^{o}, —C(O)H, —C(O)R^{o}, —C(O)NHR^{o}, —OC(O)R^{o}, —S(O)₂R^{o}, —SO₂NH₂, —S(O)R^{o} or —NHSO₂R^{o}. R^{o} is as defined above for heteroaryl groups.

The term “alkyl”, as used herein, unless otherwise indicated, includes straight, branched or cyclic saturated monovalent hydrocarbon radicals, typically C1-C10, preferably C1-C6. Examples of alkyl groups include, but are not limited to, methyl, ethyl, propyl, isopropyl, and t-butyl.

The term “haloalkyl”, as used herein, includes an alkyl substituted with one or more F, Cl, Br, or I, wherein the alkyl is defined above.

The terms “alkoxy”, as used herein, means an “alkyl-O—” group, wherein alkyl, is defined above.

It has been shown that HMGB polypeptides (e.g., HMGB1) bind Toll-like receptor 2 (TLR2) and that inhibition of the interaction between HMGB and TLR2 can decrease or prevent inflammation (U.S. Published Application No. 20040053841; the entire teachings of which are incorporated herein by reference). Therefore, agents that bind to HMGB and inhibit interaction between HMGB and TLR2 (e.g., antibodies to HMGB, antibodies to HMGB B boxes (as described herein), HMGB small molecule antagonists), as well as agents that bind to TLR2 and inhibit interaction between HMGB and TLR2 (e.g., antibodies to TLR2, TLR2 small molecule antagonists, soluble TLR2) are encompassed by the invention.

In one embodiment, the combination therapy composition comprises an agent that binds to TLR2 and inhibits interaction between HMGB and TLR2. Such agents include, e.g., an antibody or antigen-binding fragment that binds TLR2, a mutant of a natural ligand, a peptidomimetic, a competitive inhibitor of ligand binding). In one embodiment, the agent is a ligand that binds to TLR2 with greater affinity than HMGB binds to TLR2. Preferably the agent that binds to TLR2, thereby inhibiting binding by HMGB, does not significantly initiate or increase an inflammatory response, and/or does not significantly initiate or increase the release of a proinflammatory cytokine from a cell.

Examples of ligands that are known to bind TLR2 include heat shock protein 60, surfactant protein-A, monophosphoryl lipid A (Botler et al., Infect. Immun. 71(5): 2498-2507 (2003)), muramyl dipeptide (Beutler et al., Blood Cells Mol. Dis. 27(4):728-730 (2001)), yeast-particle zymosan, GPI anchor from Trypanosoma cruzi, Listeria monocytogenes, Bacillus, lipoteichoic acid, peptidoglycan, and lipopeptides from Streptococcus species, heat killed Mycobacteriua tuberculosis, Mycobacteria avium lipopeptide, lipoarabinomannan, mannosylated phosphatidylinositol, Borrelia burgdorferi, Treponema pallidum, Treponema maltophilum (lipopeptides, glycolipids, outer surface protein A), and MALP-2 lipopeptides from Mycoplasma fermentans. Therefore, these molecules, as well as portions of these molecules that bind TLR2 can be used to inhibit the interaction between HMGB and TLR2 and can be used in the combination therapy compositions and methods of the invention.

In another embodiment, the combination therapy composition comprises an agent that binds to HMGB, and prevents HMGB from binding to TLR2. Such an agent can be, for example, a soluble form of recombinant TLR2 (sTLR2) (i.e., TLR2 lacking the intracellular and transmembrane domains, as described, for example, by Iwaki et al., J. Biol. Chem. 277(27):24315-24320 (2002)), an anti-HMGB antibody or antigen-binding fragment (as described herein), or a non-HMGB antibody molecule (e.g., a protein, peptide, or small molecule antagonist) that binds HMGB and prevents it from binding to TLR2. The sTLR2 molecule can contain the extracellular domain (for example, amino acids 1-587 of the TLR2 amino acid sequence (e.g., GenBank Accession Number AAC34133)). The sTLR molecule can also be modified with one of more amino acid substitutions and/or post-translational modifications provided such sTLR2 molecules have HMGB binding activity, which can be assessed using methods known in the art. Such sTLR2 molecules can be made, for example, using recombinant techniques. Preferably the sTLR2 has at least 70%, 75%, 80%, 85%, 90%, or 95%, to amino acids 1-587 of GenBank Accession Number AAC34133. In another embodiment, the inhibitor is an agent that bind TLR2 at a site different than the HMGB binding site and blocks binding by HMGB (e.g., by causing a conformation change in the TLR2 protein or otherwise altering the binding site for HMGB). In another embodiment, the combination therapy composition comprises a dominant negative mutant protein of TLR2 and an agent that inhibits complement biological activity.

It has also been shown that receptor signal transduction of HMGB1 occurs in part through Toll-like receptor 4 (TLR4). Park, J. S. et al., J. Biol. Chem. 279(9):7370-77 (2004). Therefore, agents that bind to TLR4 and inhibit HMGB1 binding and/or signaling and/or bind to HMGB and inhibit TLR4-mediated binding and/or signaling are encompassed by the invention. Such agents include, e.g., antibodies to TLR4, TLR4 small molecule antagonists, soluble TLR4, dominant negative mutants of TLR4, mutants of a natural ligand of TLR4, peptidomimetics and competitive inhibitors of ligand binding to TLR4.

In one embodiment, the combination therapy composition comprises a soluble TLR4 and an agent that inhibits complement biological activity. It has been shown in mice that there is an alternatively spliced TLR4 mRNA (mTLR4), which expresses a partially secreted 20 kDa protein (soluble mTLR4; smTLR4) that inhibits LPS-mediated TNF-α production and NF-κB activation. Iwami, K-I et al., J Iimmunol. 165:6682-6686 (2001); the entire teachings of which are incorporated herein by reference. In another embodiment, the combination therapy composition comprises an antibody that binds TLR4 or an antigen-binding fragment thereof and an agent that inhibits complement biological activity. Antibodies that bind TLR4 are known in the art. See, e.g., Tabeta, K. et al., Infect Immun. 68(6):3731-3735 (2000); Rabbit anti-TLR-4 (Catalog No. 36-3700; Zymed Laboratories, Inc., San Francisco, Calif.).

It has been shown that HMGB polypeptides bind RAGE and that receptor signal transduction occurs in part through the receptor for advanced glycation end-products (RAGE). Andersson, U. et al., Scand. J. Infect. Dis. 35(9):577-84 (2003); Park, J. S. et al., J. Biol. Chem. 279(9):7370-77 (2004). It has further been shown that inhibition of the interaction between HMGB and RAGE can decrease or prevent downstream signaling and cellular activation (Schmidt, A. M. et al., J. Clin. Invest. 108(7):949-955 (2001); Park, J. S. et al, J. Biol. Chem. 279(9):7370-77 (2004). Therefore, agents that bind to HMGB and inhibit interaction between HMGB and RAGE (e.g., antibodies to HMGB, antibodies to HMGB B boxes (as described herein), HMGB small molecule antagonists, as well as agents that bind to RAGE and inhibit interaction between HMGB and RAGE (e.g., antibodies to RAGE, RAGE small molecule antagonists (e.g., as taught in PCT Publication Nos. WO 01/99210, WO 02/069965 and WO 03/075921 and U.S. Published Application No. US 2002/0193432A1)), soluble RAGE (sRAGE; e.g., as taught in Schmidt, A. M. et al., J. Clin. Invest. 108(7):949-955 (2001), U.S. Application No. 2002/0122799 and PCT Publication No. WO 00/20621), RAGE dominant negative mutants (as taught in Schmidt, A. M. et al., J. Clin. Invest. 108(7):949-955 (2001)) are encompassed by the invention.

In one embodiment, the combination therapy composition comprises an agent that binds to RAGE and inhibits interaction between HMGB and RAGE. Such agents include, e.g., an antibody or antigen-binding fragment that binds RAGE, a mutant of a natural ligand, a peptidomimetic, a competitive inhibitor of ligand binding). In one embodiment, the agent is a ligand that binds to RAGE with greater affinity than HMGB binds to RAGE. Preferably the agent that binds to RAGE, thereby inhibiting binding by HMGB, does not significantly initiate or increase an inflammatory response, and/or does not significantly initiate or increase the release of a proinflammatory cytokine from a cell.

Examples of ligands other than HMGB1 that are known to bind RAGE include: AGEs (advanced glycation endproducts, S100/calgranulins and β-sheet fibrils. Schmidt, A. M. et al., J. Clin. Invest. 108(7):949-955 (2001)). Therefore, these molecules, as well as portions of these molecules that bind RAGE can be used to inhibit the interaction between HMGB and RAGE and can be used in the combination therapy compositions and methods of the invention.

In another embodiment, the combination therapy composition comprises an agent that binds to HMGB, and prevents HMGB from binding to RAGE. Such an agent can be, for example, a soluble truncated form of RAGE (sRAGE) (i.e., RAGE lacking its intracellular and transmembrane domains, as described, for example, by Schmidt, A. M. et al., J. Clin. Invest. 108(7):949-955 (2001), U.S. Application No. 2002/0122799 and PCT Publication No. WO 00/20621), an anti-HMGB antibody or antigen-binding fragment (as described herein), or a non-HMGB antibody molecule (e.g., a protein, peptide, or non-peptidic small molecule) that binds HMGB and prevents it from binding to RAGE. The sRAGE molecule can be modified with one of more amino acid substitutions and/or post-translational modifications provided such sRAGE molecules have HMGB binding activity, which can be assessed using methods known in the art. Such sRAGE molecules can be made, for example, using recombinant techniques. In another embodiment, the inhibitor is an agent that bind RAGE at a site different than the HMGB binding site and blocks binding by HMGB (e.g., by causing a conformation change in the RAGE protein or otherwise altering the binding site for HMGB). In another embodiment, the combination therapy composition comprises a dominant negative mutant protein of RAGE and an agent that inhibits complement biological activity. Dominant negative mutant RAGE proteins, which are capable of binding to RAGE but suppress RAGE-mediated signaling are known in the art, e.g., as described by Schmidt, A. M. et al., J. Clin. Invest. 108(7):949-955 (2001)).

In a particular embodiment, the inhibitor of HMGB receptor binding is not an anti-TLR2 antibody or antigen-binding fragment thereof. In another embodiment, the inhibitor of M4 GB receptor binding is not an antibody that binds HMGB1 (an anti-HMGB1 antibody) or an antigen-binding fragment thereof. In yet another embodiment, the inhibitor of HMGB receptor binding is not an antibody that binds HMGB (an anti-HMGB antibody) or an antigen-binding fragment thereof. In another embodiment, the inhibitor is not soluble RAGE (i.e., a portion of the RAGE receptor that binds HMGB 1).

In another embodiment, the inhibitor is non-microbial (i.e., is not a microbe, derived from a microbe, or secreted or released from a microbe). In still another embodiment, the inhibitor is a mammalian inhibitor (i.e., is a molecule that naturally exists in a mammal, is derived from a molecule that naturally exists in a mammal, or is secreted or released from a mammalian cell), for example, a human inhibitor.

In a particular embodiment, the inhibitor is a small molecule inhibitor (i.e., having a molecular weight of 1000 or less, 500 or less, 250 or less or 100 or less). In another embodiment the inhibitor is a short peptide, having, for example, 50 or fewer amino acids, 30 or fewer amino acids, 25 or fewer amino acids, 20 or fewer amino acids, 10 or fewer amino acids, or 5 or fewer amino acids.

As described herein, inhibitors of HMGB receptor binding and/or signaling also include, e.g., antisense nucleic acids and small double-stranded interfering RNA (RNA interference (RNAi)) that target HMGB, TLR2, TLR4 and/or RAGE. Antisense nucleic acids and RNAi can be used to decrease expression of a target molecule, e.g., HMGB, TLR2, TLR4, RAGE, as is known in the art.

Production and delivery of antisense nucleic acids and RNAi is known in the art (e.g., as taught in PCT Publication WO 2004/016229). In one embodiment, small double-stranded interfering RNA (RNA interference (RNAi)) can be used (e.g., RNAi that targets HMGB, TLR2, TLR4 and/or RAGE) in the compositions and methods of the invention. RNAi is a post-transcription process, in which double-stranded RNA is introduced, and sequence-specific gene silencing results, though catalytic degradation of the targeted mRNA. See, e.g., Elbashir, S. M. et al., Nature 411:494-498 (2001); Lee, N. S., Nature Biotech. 19:500-505 (2002); Lee, S-K. et al., Nature Medicine 8(7):681-686 (2002) the entire teachings of these references are incorporated herein by reference.

RNAi is used routinely to investigate gene function in a high throughput fashion or to modulate gene expression in human diseases (Chi et al., Proc. Natl. Acad. Sci. U.S.A, 100(11):6343-6346 (2003)). Introduction of long double stranded RNA leads to sequence-specific degradation of homologous gene transcripts. The long double stranded RNA is metabolized to small 21-23 nucleotide siRNA (small interfering RNA). The siRNA then binds to protein complex RISC(RNA-induced silencing complex) with dual function helicase. The helicase has RNase activity and is able to unwind the RNA. The unwound siRNA allows an antisense strand to bind to a target. This results in sequence dependent degradation of cognate mRNA. Aside from endogenous RNAi, exogenous RNAi, chemically synthesized or recombinantly produced RNAi can also be used in the compositions and methods of the invention.

In one embodiment, the methods of the invention utilize aptamers of HMGB (e.g., aptamers of HMGB1). As is known in the art, aptamers are macromolecules composed of nucleic acid (e.g., RNA, DNA) that bind tightly to a specific molecular target (e.g., an HMGB protein, an HMGB box (e.g., an HMGB A box, an HMGB B box), an HMGB polypeptide and/or an HMGB epitope). A particular aptamer may be described by a linear nucleotide sequence and is typically about 15-60 nucleotides in length. The chain of nucleotides in an aptamer form intramolecular interactions that fold the molecule into a complex three-dimensional shape, and this three-dimensional shape allows the aptamer to bind tightly to the surface of its target molecule. Given the extraordinary diversity of molecular shapes that exist within the universe of all possible nucleotide sequences, aptamers may be obtained for a wide array of molecular targets, including proteins and small molecules. In addition to high specificity, aptamers have very high affinities for their targets (e.g., affinities in the picomolar to low nanomolar range for proteins). Aptamers are chemically stable and can be boiled or frozen without loss of activity. Because they are synthetic molecules, they are amenable to a variety of modifications, which can optimize their function for particular applications. For example, aptamers can be modified to dramatically reduce their sensitivity to degradation by enzymes in the blood for use in in vivo applications. In addition, aptamers can be modified to alter their biodistribution or plasma residence time.

Selection of aptamers that can bind HMGB or a fragment thereof (e.g., HMGB1 or a fragment thereof) can be achieved through methods known in the art. For example, aptamers can be selected using the SELEX (Systematic Evolution of Ligands by Exponential Enrichment) method (Tuerk, C., and Gold, L., Science 249:505-510 (1990)). In the SELEX method, a large library of nucleic acid molecules (e.g., 10¹⁵ different molecules) is produced and/or screened with the target molecule (e.g., an HMGB protein, an HMGB box (e.g., an HMGB A box, an HMGB B box), an HMGB polypeptide and/or an HMGB epitope). The target molecule is allowed to incubate with the library of nucleotide sequences for a period of time. Several methods, known in the art, can then be used to physically isolate the aptamer target molecules from the unbound molecules in the mixture, which can be discarded. The aptamers with the highest affinity for the target molecule can then be purified away from the target molecule and amplified enzymatically to produce a new library of molecules that is substantially enriched for aptamers that can bind the target molecule. The enriched library can then be used to initiate a new cycle of selection, partitioning, and amplification. After 5-15 cycles of this iterative selection, partitioning and amplification process, the library is reduced to a small number of aptamers that bind tightly to the target molecule. Individual molecules in the mixture can then be isolated, their nucleotide sequences determined, and their properties with respect to binding affinity and specificity measured and compared. Isolated aptamers can then be further refined to eliminate any nucleotides that do not contribute to target binding and/or aptamer structure, thereby producing aptamers truncated to their core binding domain. See Jayasena, S. D. Clin. Chem. 45:1628-1650 (1999) for review of aptamer technology; the entire teachings of which are incorporated herein by reference).

In particular embodiments, the methods of the invention utilize aptamers having the same or similar binding specificity as described herein for HMGB antagonists (e.g., binding specificity for an HMGB polypeptide, fragment of an HMGB polypeptide (e.g., an HMGB A box, an HMGB B box), epitopic region of an HMGB polypeptide). In particular embodiments, the aptamers of the invention can bind to an HMGB polypeptide or fragment thereof and inhibit one or more functions of the HMGB polypeptide. As described herein, functions of HMGB polypeptides include, e.g., increasing inflammation, increasing release of a proinflammatory cytokine from a cell, binding to RAGE, binding to TLR2, chemoattraction. In a particular embodiment, the aptamer binds HMGB1 (e.g., human HMGB1) or a fragment thereof (e.g., an A box, a B box) and inhibits one or more functions of the HMGB polypeptide (e.g., inhibits release of a proinflammatory cytokine from a vertebrate cell treated with HMGB).

Inhibitors of Complement Biological Activity

The complement system acts in conjunction with other immunological systems of the body to defend against intrusion of cellular and viral pathogens. There are at least 25 complement proteins, which are found as a complex collection of plasma proteins and membrane cofactors. The plasma proteins make up about 10% of the globulins in vertebrate serum. Complement components achieve their immune defensive functions by interacting in a series of intricate but precise enzymatic cleavage and membrane binding events. The resulting complement cascade leads to the production of products with opsonic, immunoregulatory, and lytic functions.

The complement cascade progresses via the classical pathway or the alternative pathway. The classical complement pathway is typically initiated by antibody recognition of, and binding to, an antigenic site on a target cell. The alternative pathway is usually antibody independent, and can be initiated by certain molecules on pathogen surfaces. Both pathways converge at the point where complement component C3 is cleaved by an active protease (which is different in each pathway) to yield C3a and C3b. C3b, together with the same “terminal complement” components (C5 through C9), common to both pathways, are responsible for the activation and destruction of target cells through initiation of the membrane attack complex (MAC). The MAC causes osmotic lysis of the pathogen. Proteolytic activation of components C3, C4, and C5 leads to release of the anaphylatoxins C3a, C4a, and C5a, which have chemotactic effects on inflammatory cells. Overall, complement activation can result in one or more of the following biological activities: cell lysis, degranulation of mast cells and basophils, extravasation and chemotaxis of neutrophils and monocytes, opsonization of antigens, viral neutralization, and clearance of immune complexes.

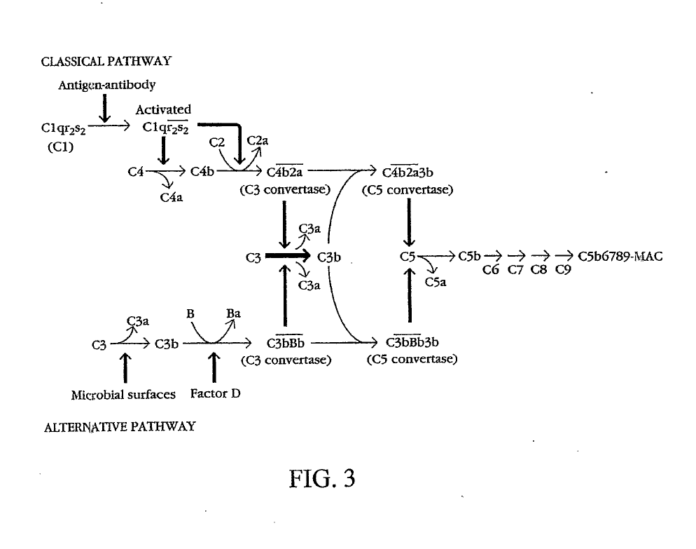

FIG. 3 provides an overview of the complement activation pathways. As described above, the classical pathway is initiated by binding of C1 to antigen-antibody complexes. The alternative pathway is initiated by binding of C3b to activating surfaces such as pathogens. Both pathways generate C3 and C5 convertases. C5 convertase acts to cleave the substrate into C5a and C5b, and C5b is converted into a MAC by a common sequence of terminal reactions. Hydrolysis of C3 is the major amplification step in both pathways, generating large amounts of C3b, which forms part of C5 convertase.

While the complement system is powerful in protecting the body from invading pathogens, inappropriate activation of the complement system has been associated with a number of diseases and conditions, including inflammatory conditions. One method for treating such inflammatory conditions is to inhibit complement biological activity.

As used herein, “an agent that inhibits complement biological activity” is an agent that decreases one or more of the biological activities of the complement system. Examples of complement biological activity include, but are not limited to, cell lysis, development of an inflammatory response, opsonization of antigen, viral neutralization, and clearance of immune complexes. Components of the complement system participate in the development of an inflammatory response by degranulating mast cells, basophils, and eosinophils, aggregation of platelets, and release of neutrophils from bone marrow. Agents that inhibit complement biological activity include, e.g., agents that inhibit (decrease) the interaction between a complement component and its receptor(s), agents that inhibit (decrease) formation of the MAC, agents that inhibit a key protein in the complement cascade, agents that inhibit conversion of complement C5 to C5a and C5b, and agents that inhibit the action of complement-derived anaphalytoxins C3a and C5a. Such agents include, but are not limited to peptides, proteins, synthesized molecules (for example, synthetic organic molecules), naturally-occurring molecule (for example, naturally occurring organic molecules), nucleic acid molecules, and components thereof. Preferred examples of agents that inhibit complement biological activity include agents that inhibit expression or activity or one or more of the following components of the complement system: C1q, C1r, C1s, Factor D, Factor B, Properdin, C2, C3, C4, C5, C6, C7, C8, C9, C3 convertase, C5 convertase, as well as fragments of components that are produced upon activation of complement, for example, fragment 2a, 2b, 3a, 3b, 4a, 4b, 5a, and/or 5b.

Examples of agents that inhibit complement biological activity include, but are not limited to: C5 inhibitors, for example, 5G1.1 (also known as Eculizumab; Alexion Pharmaceuticals, Inc., Cheshire, Conn.) and h5G1.1-SC (also known as Pexelizumab, Alexion Pharmaceuticals Inc., Cheshire, Conn.); C5a receptor antagonists, for example, NGD 2000-1 (Neurogen, Corp., Branford, Conn.) and AcPhe[Orn-Pro-D-Cyclohexylalanine-Trp-Arg] (AcF-[OPdChaWR]; see, e.g., Strachan, A. J. et al., Br. J. Pharmacol. 134(8):1778-1786 (2001)); C1 esterase inhibitor (C1-INH); Factor H (inactive C3b); Factor I (inactive C4b); soluble complement receptor type 1 (sCR1; see, e.g., U.S. Pat. No. 5,856,297) and sCR1-sLe(X) (see, e.g., U.S. Pat. No. 5,856,300; membrane cofactor protein (MCP), decay accelerating factor (DAF) and CD59 and soluble recombinant forms thereof (Ashgar, S. S. et al., Front Biosci. 5:E63-E81 (2000) and Sohn, J. H. et al., Invest. Opthamol. Vis. Sci. 41(13):4195-4202 (2000)); Compstatin (Morikis et al., Protein Sci. 7:619-627 (1998); Sahu, A. et al., J. Immunol. 165:2491-2499 (2000)); chimeric complement inhibitor proteins having at least two complementary inhibitory domains (see, e.g., U.S. Pat. Nos. 5,679,546, 5,851,528 and 5,627,264); and small molecule antagonists (see, e.g., PCT Publication No. WO 02/49993, U.S. Pat. Nos. 5,656,659, 5,652,237, 4,510,158, 4,599,203 and 4,231,958). Other known complement inhibitors are known in the art and are encompassed by the invention. In addition, methods for measuring complement activity (e.g., to identify agents that inhibit complement activity) are known in the art. Such methods include, e.g., using a 50% hemolytic complement (CH₅₀) assay (see, e.g., Kabat et al., Experimental Immunochemistry, 2nd Ed. (Charles C. Thomas, Publisher, Springfield, Ill.), p. 133-239 (1961)), using an enzyme immunoassay (EIA), using a liposome immunoassay (LIA) (see, e.g., Jaskowski et al., Clin. Diagn. Lab. Immunol. 6(1):137-139 (1999)).

Compositions Comprising an HMGB a Box Polypeptide and/or an Antibody to HMGB and an Inhibitor of Complement Biological Activity

The present invention is directed to a composition comprising any of the above-described HMGB A box polypeptides, and/or an antibody or antigen binding fragment thereof that binds HMGB, and/or an antibody or antigen binding fragment thereof that binds an HMGB B box and/or an antibody or antigen binding fragment thereof that binds an HMGB A box, and an agent that inhibits complement biological activity (collectively termed “combination therapy compositions”). In one embodiment, the combination therapy composition comprises an HMGB A box polypeptide, or an antibody or antigen binding fragment thereof that binds HMGB, or an antibody or antigen binding fragment thereof that binds an HMGB B box, or an antibody or antigen binding fragment thereof that binds an HMGB A box, and an agent that inhibits complement biological activity. Alternatively, the combination therapy composition can comprise more than one HMGB A box polypeptide, and/or antibody or antigen binding fragment thereof that binds HMGB, and/or antibody or antigen binding fragment thereof that binds an HMGB B box and/or an antibody or antigen binding fragment thereof that binds an HMGB A box, and an agent that inhibits complement biological activity.

Preferred examples of agents that inhibit complement biological activity include C5 inhibitors, for example, 5G1.1 (also known as Eculizumab) and h5G1.1-SC (also known as Pexelizumab); C5a receptor antagonists, for example, NGD 2000-1 and AcPhe[Om-Pro-D-Cyclohexylalanine-Trp-Arg] (AcF-[OPdChaWR]); C1 esterase inhibitor (C1-INH); Factor H (inactive C3b); Factor I (inactive C4b); soluble complement receptor type 1 (sCR1) inhibitor and sCR1-sLe(X); membrane cofactor protein (MCP), decay accelerating factor (DAF) and CD59 and soluble recombinant forms thereof; Compstatin; chimeric complement inhibitor proteins having at least two complementary inhibitory domains; and small molecule antagonists. Such combination therapy compositions can further comprise a pharmaceutically acceptable carrier.

Treatment of Inflammatory Conditions

The present invention provides a method of treating an inflammatory condition in an individual, or treating an individual at risk for having an inflammatory condition, comprising administering to the individual an effective amount of a combination therapy composition as described herein. As used herein, an “effective amount” is an amount sufficient to prevent or decrease an inflammatory response, and/or to ameliorate and/or decrease the longevity of symptoms associated with an inflammatory response. Methods for determining whether a combination therapy composition inhibits an inflammatory condition are known to one skilled in the art. Inhibition of the release of a proinflammatory cytokine from a cell can be measured by any method known to one of skill in the art, for example, using the L929 cytotoxicity assay described herein.

The inflammatory condition can be one in which the inflammatory cytokine cascade is activated. In one embodiment, the inflammatory cytokine cascade causes a systemic reaction, such as with endotoxic shock. In another embodiment, the inflammatory condition is mediated by a localized inflammatory cytokine cascade, as in rheumatoid arthritis. Other nonlimiting examples of inflammatory conditions that can be usefully treated using the present invention include those described herein.

In one embodiment, the condition to be treated is selected from one or more of the group consisting of sepsis, peritonitis, pancreatitis, inflammatory bowel disease, ileus, ulcerative colitis, Crohn's disease, ischemia, for example, myocardial ischemia, organic ischemia, or reperfusion ischemia, cachexia, burns, adult respiratory distress syndrome, multiple sclerosis, atherosclerosis, restenosis, arthritis, rheumatoid arthritis, asthma, lupus, adult respiratory distress syndrome, chronic obstructive pulmonary disease, psoriasis, Behcet's syndrome, psoriasis, allograft rejection and graft-versus-host disease. Where the condition is allograft rejection, the composition may advantageously also include an immunosuppressant that is used to inhibit allograft rejection, such as cyclosporin.

Preferably the combination therapy compositions are administered to a patient in need thereof in an amount sufficient to inhibit release of proinflammatory cytokine from a cell and/or to treat an inflammatory condition. In one embodiment, release of the proinflammatory cytokine is inhibited by at least 10%, 20%, 25%, 50%, 75%, 80%, 90%, or 95%, as assessed using methods described herein or other methods known in the art.

The terms “therapy,” “therapeutic,” and “treatment” as used herein, refer to ameliorating symptoms associated with a disease or condition, for example, an inflammatory disease or an inflammatory condition, including preventing or delaying the onset of the disease symptoms, and/or lessening the severity or frequency of symptoms of the disease or condition. The terms “subject” and “individual” are defined herein to include animals such as mammals, including, but not limited to, primates, cows, sheep, goats, horses, dogs, cats, rabbits, guinea pigs, rats, mice or other bovine, ovine, equine, canine, feline, rodent, or murine species. In one embodiment, the animal is a human.

The carrier included with the combination therapy compositions is chosen based on the expected route of administration of the composition in therapeutic applications. The route of administration of the composition depends on the condition to be treated. For example, intravenous injection may be preferred for treatment of a systemic disorder such as endotoxic shock, and oral administration may be preferred to treat a gastrointestinal disorder such as a gastric ulcer. The dosage of the combination therapy compositions to be administered can be determined by the skilled artisan without undue experimentation in conjunction with standard dose-response studies. Relevant circumstances to be considered in making those determinations include the condition or conditions to be treated, the choice of composition to be administered, the age, weight, and response of the individual patient, and the severity of the patient's symptoms. Typically, an effective amount can range from 0.01 mg per day to about 100 mg per day for an adult. Preferably, the dosage ranges from about 1 mg per day to about 100 mg per day or from about 1 mg per day to about 10 mg per day. Depending on the condition, the combination therapy composition can be administered orally, parenterally, intranasally, vaginally, rectally, lingually, sublingually, buccally, intrabuccally and/or transdermally to the patient.

Accordingly, combination therapy compositions designed for oral, lingual, sublingual, buccal and intrabuccal administration can be made without undue experimentation by means well known in the art, for example, with an inert diluent or with an edible carrier. The combination therapy composition may be enclosed in gelatin capsules or compressed into tablets. For the purpose of oral therapeutic administration, the pharmaceutical compositions of the present invention may be incorporated with excipients and used in the form of tablets, troches, capsules, elixirs, suspensions, syrups, wafers, chewing gums, and the like.

Tablets, pills, capsules, troches and the like may also contain binders, recipients, disintegrating agent, lubricants, sweetening agents, and/or flavoring agents. Some examples of binders include microcrystalline cellulose, gum tragacanth and gelatin. Examples of excipients include starch and lactose. Some examples of disintegrating agents include alginic acid, corn starch, and the like. Examples of lubricants include magnesium stearate and potassium stearate. An example of a glidant is colloidal silicon dioxide. Some examples of sweetening agents include sucrose, saccharin, and the like. Examples of flavoring agents include peppermint, methyl salicylate, orange flavoring, and the like. Materials used in preparing these various compositions should be pharmaceutically pure and non-toxic in the amounts used.

The combination therapy compositions of the present invention can be administered parenterally, such as, for example, by intravenous, intramuscular, intrathecal and/or subcutaneous injection. Parenteral administration can be accomplished by incorporating the combination therapy compositions of the present invention into a solution or suspension. Such solutions or suspensions may also include sterile diluents, such as water for injection, saline solution, fixed oils, polyethylene glycols, glycerine, propylene glycol and/or other synthetic solvents. Parenteral formulations may also include antibacterial agents, such as, for example, benzyl alcohol and/or methyl parabens, antioxidants, such as, for example, ascorbic acid and/or sodium bisulfite, and chelating agents, such as EDTA. Buffers, such as acetates, citrates and phosphates, and agents for the adjustment of tonicity, such as sodium chloride and dextrose, may also be added. The parenteral preparation can be enclosed in ampules, disposable syringes and/or multiple dose vials made of glass or plastic.

Rectal administration includes administering the combination therapy composition into the rectum and/or large intestine. This can be accomplished using suppositories and/or enemas. Suppository formulations can be made by methods known in the art. For example, suppository formulations can be prepared by heating glycerin to about 120° C., dissolving the combination therapy composition in the glycerin, mixing the heated glycerin after which purified water may be added, and pouring the hot mixture into a suppository mold.

Transdermal administration includes percutaneous absorption of the composition through the skin. Transdermal formulations include patches, ointments, creams, gels, salves, and the like.

The combination therapy compositions of the present invention can be administered nasally to a patient. As used herein, nasally administering or nasal administration includes administering the combination therapy compositions to the mucous membranes of the nasal passage and/or nasal cavity of the patient. Pharmaceutical compositions for nasal administration of a composition include therapeutically effective amounts of the combination therapy composition prepared by well-known methods to be administered, for example, as a nasal spray, nasal drop, suspension, gel, ointment, cream and/or powder. Administration of the composition may also take place using a nasal tampon and/or nasal sponge.

If desired, the combination therapy compositions described herein can also include one or more additional agents used to treat an inflammatory condition. Such agents are known to one of skill in the art. The agent may be, for example, an antagonist of an early sepsis mediator. As used herein, an early sepsis mediator is a proinflammatory cytokine that is released from cells soon (i.e., within 30-60 min.) after induction of an inflammatory cytokine cascade (e.g., exposure to LPS). Nonlimiting examples of these cytokines are IL-1α, IL-1β, IL-6, PAF, and MIF. Also included as early sepsis mediators are receptors for these cytokines (for example, tumor necrosis factor receptor type 1) and enzymes required for production of these cytokines (for example, interleukin-1β converting enzyme). Antagonists of any early sepsis mediator, now known or later discovered, can be useful for these embodiments by further inhibiting an inflammatory cytokine cascade.

Nonlimiting examples of antagonists of early sepsis mediators are antisense compounds that bind to the mRNA of the early sepsis mediator, preventing its expression (see, e.g., Ojwang et al., Biochemistry 36:6033-6045, 1997; Pampfer et al., Biol. Reprod. 52:1316-1326, 1995; U.S. Pat. No. 6,228,642; Yahata et al., Antisense Nucleic Acid Drug Dev. 6:55-61, 1996; and Taylor et al., Antisense Nucleic Acid Drug Dev. 8:199-205, 1998), ribozymes that specifically cleave the mRNA of the early sepsis mediator (see, e.g., Leavitt et al., Antisense Nucleic Acid Drug Dev. 10:409-414, 2000; Kisich et al., 1999; and Hendrix et al., Biochem. J. 314 (Pt. 2):655-661, 1996), and antibodies that bind to the early sepsis mediator and inhibit their action (see, e.g., Kam and Targan, Expert Opin. Pharmacother. 1:615-622, 2000; Nagahira et al., J. Immunol. Methods 222:83-92, 1999; Lavine et al., J. Cereb. Blood Flow Metab. 18:52-58, 1998; and Hohnes et al, Hybridoma 19:363-367, 2000). An antagonist of an early sepsis mediator, now known or later discovered, is envisioned as within the scope of the invention. The skilled artisan can determine the amount of early sepsis mediator to use in these compositions for inhibiting any particular inflammatory cytokine cascade without undue experimentation, e.g., using routine dose-response studies.

Other agents that can be administered with the combination therapy compositions described herein include, e.g., Vitaxin™ and other antibodies targeting αᵥβ₃ integrin (see, e.g., U.S. Pat. No. 5,753,230, PCT Publication Nos. WO 00/78815 and WO 02/070007; the entire teachings of all of which are incorporated herein by reference) and anti-IL-9 antibodies (see, e.g., PCT Publication No. WO 97/08321; the entire teachings of which are incorporated herein by reference).

In one embodiment, the combination therapy compositions of the invention are administered with inhibitors of TNF biological activity. Such inhibitors of TNF activity include, e.g., peptides, proteins, synthesized molecules, for example, synthetic organic molecules, naturally-occurring molecule, for example, naturally occurring organic molecules, nucleic acid molecules, and components thereof. Preferred examples of agents that inhibit TNF biological activity include infliximab (REMICADE®; Centocor, Inc., Malvern, Pa.), etanercept (ENBREL®; Immunex; Seattle, Wash.), adalimumab (HUMIRA®; D2E7; Abbot Laboratories, Abbot Park Ill.), CDP870 (Pharmacia Corporation; Bridgewater, N.J.) CDP571 (Celltech Group plc, United Kingdom), Lenercept (Roche, Switzerland), and Thalidomide.

The relevant teachings of all publications cited herein not previously incorporated by reference, are incorporated herein by reference in their entirety. While this invention has been particularly shown and described with references to preferred embodiments thereof, it will be understood by those skilled in the art that various changes in form and details may be made therein without departing from the scope of the invention encompassed by the appended claims.

What is claimed is:

1. A pharmaceutical composition comprising a polypeptide and an agent that inhibits complement biological activity, wherein said polypeptide comprises a high mobility group box (HMGB) A box or a biologically active fragment thereof.

2. The pharmaceutical composition of claim 1, wherein said HMGB A box or biologically active fragment thereof is a mammalian HMGB A box or biologically active fragment thereof.

3. The pharmaceutical composition of claim 2, wherein said mammalian HMGB A box or biologically active fragment thereof is a mammalian HMGB1 A box or biologically active fragment thereof.

4. The pharmaceutical composition of claim 3, wherein said mammalian HMGB1 A box or biologically active fragment thereof comprises SEQ ID NO:4.

5. The pharmaceutical composition of claim 3, wherein said mammalian HMGB1 A box or biologically active fragment thereof consists of SEQ ID NO:4.

6. The pharmaceutical composition of claim 1, wherein said agent that inhibits complement biological activity is selected from the group consisting of: a C5 inhibitor, a C5a receptor antagonist, a C1 esterase inhibitor, Factor H, Factor I, a soluble complement receptor type 1 (sCR1), a sCR1-sLe(X), membrane cofactor protein (MCP) or a soluble recombinant form thereof, decay accelerating factor (DAF) or a soluble recombinant form thereof, CD59 or a soluble recombinant form thereof, Compstatin, a chimeric complement inhibitor protein comprising at least two complementary inhibitory domains, and a small molecule antagonist.

7. The pharmaceutical composition of claim 6, wherein said agent that inhibits complement biological activity is a C5 inhibitor selected from the group consisting of 5G1.1 and h5G1.1-SC.

8. The pharmaceutical composition of claim 6, wherein said agent that inhibits complement biological activity is a C5a receptor antagonist.

9. The pharmaceutical composition of claim 8, wherein said C5a receptor antagonist is NGD 2000-1.

10. The pharmaceutical composition of claim 6, wherein said agent that inhibits complement biological activity is a sCR1.

11. The pharmaceutical composition of claim 1, wherein said composition further comprises a pharmaceutically acceptable carrier.

12. A pharmaceutical composition comprising an antibody or antigen-binding fragment thereof that binds an HMGB polypeptide or a fragment thereof and an agent that inhibits complement biological activity.

13. The pharmaceutical composition of claim 12, wherein said HMGB polypeptide or fragment thereof is a mammalian HMGB polypeptide or fragment thereof.

14. The pharmaceutical composition of claim 12, wherein said HMGB polypeptide or fragment thereof is an HMGB1 polypeptide or fragment thereof.

15. The pharmaceutical composition of claim 14, wherein said HMGB1 polypeptide or fragment thereof consists of SEQ ID NO:1.

16. The pharmaceutical composition of claim 12, wherein said HMGB polypeptide or fragment thereof is an HMGB B box or biologically active fragment thereof.

17. The pharmaceutical composition of claim 16, wherein said HMGB B box or biologically active fragment thereof is an HMGB B box consisting of SEQ ID NO:5.

18. The pharmaceutical composition of claim 16, wherein said HMGB B box or biologically active fragment thereof is a biologically active fragment consisting of SEQ ID NO:45.

19. The pharmaceutical composition of claim 12, wherein said HMGB polypeptide or fragment thereof is an HMGB A box or biologically active fragment thereof

20. The pharmaceutical composition of claim 19, wherein said HMGB A box or fragment thereof is an HMGB A box consisting of SEQ ID NO:4.

21. The pharmaceutical composition of claim 12, wherein said antibody or antigen-binding fragment thereof is a monoclonal antibody or an antigen-binding fragment of a monoclonal antibody.

22. The pharmaceutical composition of claim 12, wherein said antibody or antigen-binding fragment thereof is a polyclonal antibody or an antigen-binding fragment of a polyclonal antibody.

23. The pharmaceutical composition of claim 12, wherein said agent that inhibits complement biological activity is selected from the group consisting of: a C5 inhibitor, a C5a receptor antagonist, a C1 esterase inhibitor, Factor H, Factor I, a soluble complement receptor type 1 (sCR1), a sCR1-sLe(X) cofactor protein, membrane cofactor protein (MCP) or a soluble recombinant form thereof, decay accelerating factor (DAF) or a soluble recombinant form thereof, CD59 or a soluble recombinant form thereof, Compstatin, a chimeric complement inhibitor protein comprising at least two complementary inhibitory domains, and a small molecule antagonist.

24. The pharmaceutical composition of claim 23, wherein said agent that inhibits complement biological activity is a C5 inhibitor selected from the group consisting of 5G1.1 and h5G1.1-SC.

25. The pharmaceutical composition of claim 23, wherein said agent that inhibits complement biological activity is a C5a receptor antagonist.

26. The pharmaceutical composition of claim 25, wherein said C5a receptor antagonist is NGD 2000-1.

27. The pharmaceutical composition of claim 23, wherein said agent that inhibits complement biological activity is a sCR1.

28. The pharmaceutical composition of claim 12, wherein said composition further comprises a pharmaceutically acceptable carrier.

29. A pharmaceutical composition comprising an inhibitor of HMGB receptor binding and/or HMGB signaling and an agent that inhibits complement biological activity.

30. The pharmaceutical composition of claim 29, wherein said inhibitor of HMGB receptor binding and/or HMGB signaling is an inhibitor of HMGB1 receptor binding.

31. The pharmaceutical composition of claim 29, wherein said inhibitor of HMGB receptor binding and/or HMGB signaling is selected from the group consisting of an antibody to HMGB or an antigen-binding fragment thereof, an HMGB small molecule antagonist, an antibody to TLR2 or an antigen-binding fragment thereof, a soluble TLR2 polypeptide, an antibody to RAGE or an antigen-binding fragment thereof, a soluble RAGE polypeptide and a RAGE small molecule antagonist.

32. The pharmaceutical composition of claim 31, wherein said inhibitor of HMGB receptor binding and/or HMGB signaling is an HMGB small molecule antagonist.

33. The pharmaceutical composition of claim 32, wherein said HMGB small molecule antagonist is an ester of an alpha-ketoalkanoic acid.

34. The pharmaceutical composition of claim 33, wherein said ester of an alpha-ketoalkanoic acid is an ester of a C3 to C8, straight chain or branched alpha-ketoalkanoic acid.

35. The pharmaceutical composition of claim 33, wherein said ester of an alpha-ketoalkanoic acid is an ester of pyruvic acid.

36. The pharmaceutical composition of claim 33, wherein said ester of an alpha-ketoalkanoic acid is selected from the group consisting of an ethyl ester, a propyl ester, a butyl ester, a carboxymethyl ester, an acetoxymethyl ester, a carbethoxymethyl ester and an ethoxymethyl ester.

37. The pharmaceutical composition of claim 36, wherein said ester of an alpha-ketoalkanoic acid is ethyl pyruvate.

38. The pharmaceutical composition of claim 32, wherein said HMGB small molecule antagonist is a compound represented by Formula (I a) or a pharmaceutically acceptable salt thereof: 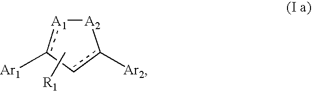
wherein:
Ar₁ and Ar₂ are independently a monocyclic six-member optionally substituted heteroaryl group;
A₁ is ═N— or —NR^{a}- and A₂ is O or S;
R^{a} is H or C1-C6 alkyl;
R₁ is selected from —H, C1-C6 alkyl, phenyl, C1-C6 haloalkyl, halogen, —OH, —OR^{b}, C1-C6 hydroxyalkyl, C1-C6 alkoxyalkyl, —O(C1-C6 haloalkyl), —SH, —SR^{b}, —NO₂, —CN, —NR^{b}CO₂R^{b}, —NR^{b}C(O)R^{b}, —CO₂R^{b}, —C(O)R^{b}, —C(O)N(R^{b})₂, —OC(O)R^{b} and —NR^{b}R^{b}; and
each R^{b} is H or a C1-C6 alkyl group.

39. The pharmaceutical composition of claim 38 wherein said compound is selected from the group consisting of: 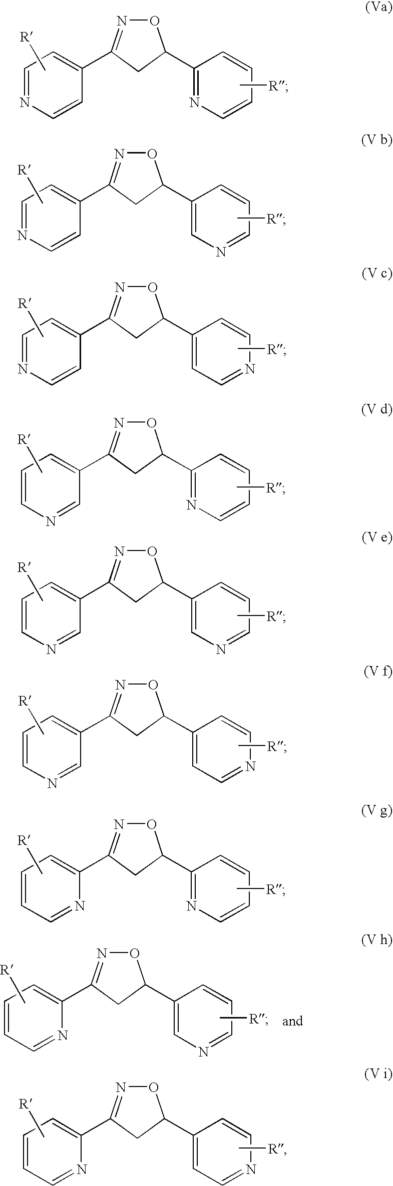
wherein:
R′ and R″ are independently —H, halogen, —NO₂, —CN, C1-C3 alkyl, C1-C3 haloalkyl, C1-C3 hydroxyalkyl, C1-C3 alkoxyalkyl, —N(R^{d})₂, —NR^{d}C(O)R^{d}, or —C(O)N(R^{d})₂.

40. The pharmaceutical composition of claim 39 wherein said compound is selected from the group consisting of:

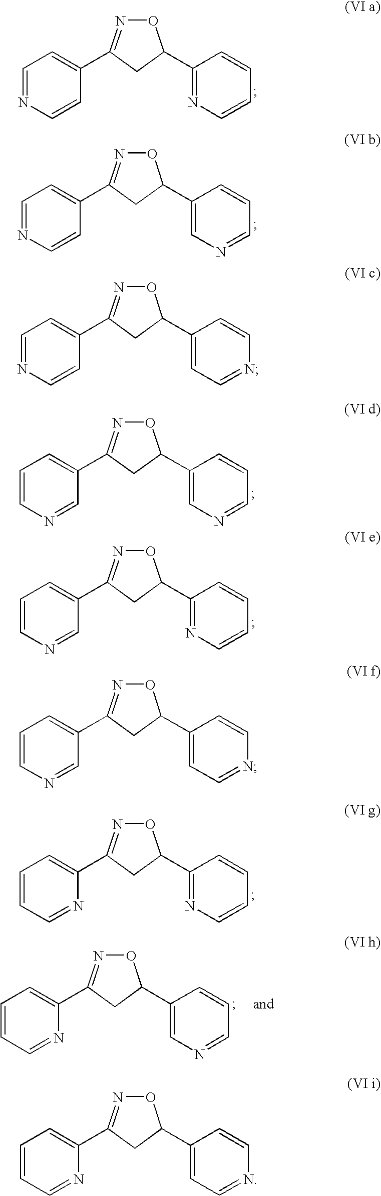

41. The pharmaceutical composition of claim 40 wherein said compound is represented by Formula (VI d):

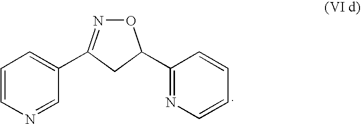

42. The pharmaceutical composition of claim 41 wherein said compound is represented by Formula (VIf):

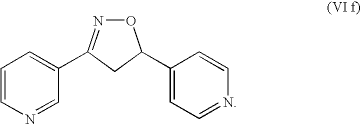

43. The pharmaceutical composition of claim 29, wherein said agent that inhibits complement biological activity is selected from the group consisting of: a C5 inhibitor, a C5a receptor antagonist, a C1 esterase inhibitor, Factor H, Factor I, a soluble complement receptor type 1 (sCR1), a sCR1-sLe(X) cofactor protein, membrane cofactor protein (MCP) or a soluble recombinant form thereof, decay accelerating factor (DAF) or a soluble recombinant form thereof, CD59 or a soluble recombinant form thereof, Compstatin, a chimeric complement inhibitor protein comprising at least two complementary inhibitory domains, and a small molecule antagonist.

44. The pharmaceutical composition of claim 43, wherein said agent that inhibits complement biological activity is a C5 inhibitor selected from the group consisting of 5G1.1 and h5G1.1-SC.

45. The pharmaceutical composition of claim 43, wherein said agent that inhibits complement biological activity is a C5a receptor antagonist.

46. The pharmaceutical composition of claim 45, wherein said C5a receptor antagonist is NGD 2000-1.

47. The pharmaceutical composition of claim 43, wherein said agent that inhibits complement biological activity is a sCR1.

48. The pharmaceutical composition of claim 29, wherein said composition further comprises a pharmaceutically acceptable carrier.

49. A method of treating an inflammatory condition in a patient comprising administering to said patient a composition comprising a polypeptide and an agent that inhibits complement biological activity, wherein said polypeptide comprises a high mobility group box (HMGB) A box or a biologically active fragment thereof.

50. The method of claim 49, wherein said composition further comprises a pharmaceutically acceptable carrier.

51. The method of claim 49, wherein said HMGB A box or biologically active fragment thereof is a mammalian HMGB A box or biologically active fragment thereof.

52. The method of claim 51, wherein said mammalian HMGB A box or biologically active fragment thereof is a mammalian HMGB1 A box or biologically active fragment thereof.

53. The method of claim 52, wherein said mammalian HMGB1 A box or biologically active fragment thereof comprises SEQ ID NO:4.

54. The method of claim 52, wherein said mammalian HMGB1 A box or biologically active fragment thereof consists of SEQ ID NO:4.

55. The method of claim 49, wherein said agent that inhibits complement biological activity is selected from the group consisting of: a C5 inhibitor, a C5a receptor antagonist, a C1 esterase inhibitor, Factor H, Factor I, a soluble complement receptor type 1 (sCR1), a sCR1-sLe(X) cofactor protein, membrane cofactor protein (MCP) or a soluble recombinant form thereof, decay accelerating factor (DAF) or a soluble recombinant form thereof, CD59 or a soluble recombinant form thereof, Compstatin, a chimeric complement inhibitor protein comprising at least two complementary inhibitory domains, and a small molecule antagonist.

56. The method of claim 55, wherein said agent that inhibits complement biological activity is a C5 inhibitor selected from the group consisting of 5G1.1 and h5G1.1-SC.

57. The method of claim 55, wherein said agent that inhibits complement biological activity is a C5a receptor antagonist.

58. The method of claim 57, wherein said C5a receptor antagonist is NGD 2000-1.

59. The method of claim 55, wherein said agent that inhibits complement biological activity is a sCR1.

60. The method of claim 49, wherein said inflammatory condition is selected from the group consisting of sepsis, allograft rejection, arthritis, asthma, lupus, adult respiratory distress syndrome, chronic obstructive pulmonary disease, psoriasis, pancreatitis, peritonitis, burns, ischemia, Behcet's disease, graft versus host disease, inflammatory bowel disease, Crohn's disease, ulcerative colitis, multiple sclerosis, and cachexia.

61. A method of treating an inflammatory condition in a patient comprising administering to said patient a composition comprising an antibody or an antigen-binding fragment thereof and an agent that inhibits complement biological activity, wherein said antibody or antigen-binding fragment thereof binds an HMGB polypeptide or a fragment thereof.

62. The method of claim 61, wherein said composition further comprises a pharmaceutically acceptable carrier.

63. The method of claim 61, wherein said HMGB polypeptide or fragment thereof is a mammalian HMGB polypeptide or fragment thereof.

64. The method of claim 61, wherein said HMGB polypeptide or fragment thereof is an HMGB1 polypeptide or fragment thereof.

65. The method of claim 64, wherein said HMGB1 polypeptide or fragment thereof consists of SEQ ID NO:1.

66. The method of claim 61, wherein said HMGB polypeptide or fragment thereof is an HMGB B box or a biologically active fragment thereof.

67. The method of claim 66, wherein said HMGB B box or biologically active fragment thereof is an HMGB B Box consisting of SEQ ID NO:5.

68. The method of claim 66, wherein said HMGB B box or biologically active fragment thereof is a biologically active fragment consisting of SEQ ID NO:45.

69. The method of claim 61, wherein said HMGB polypeptide or fragment thereof is an HMGB A box or a biologically active fragment thereof.

70. The method of claim 69, wherein said HMGB A box or biologically active fragment thereof is an HMGB A Box consisting of SEQ ID NO:4.

71. The method of claim 61, wherein said antibody or an antigen-binding fragment thereof is a monoclonal antibody or an antigen-binding fragment of a monoclonal antibody.

72. The method of claim 61, wherein said antibody or an antigen-binding fragment thereof is a polyclonal antibody or an antigen-binding fragment of a polyclonal antibody.

73. The method of claim 61, wherein said agent that inhibits complement biological activity is selected from the group consisting of: a C5 inhibitor, a C5a receptor antagonist, a C1 esterase inhibitor, Factor H, Factor I, a soluble complement receptor type 1 (sCR1), a sCR1-sLe(X) cofactor protein, membrane cofactor protein (MCP) or a soluble recombinant form thereof, decay accelerating factor (DAF) or a soluble recombinant form thereof, CD59 or a soluble recombinant form thereof, Compstatin, a chimeric complement inhibitor protein comprising at least two complementary inhibitory domains, and a small molecule antagonist.

74. The method of claim 73, wherein said agent that inhibits complement biological activity is a C5 inhibitor selected from the group consisting of 5G1.1 and h5G1.1-SC.

75. The method of claim 73, wherein said agent that inhibits complement biological activity is a C5a receptor antagonist.

76. The method of claim 75, wherein said C5a receptor antagonist is NGD 2000-1.

77. The method of claim 73, wherein said agent that inhibits complement biological activity is a sCR1.

78. The method of claim 61, wherein said inflammatory condition is selected from the group consisting of sepsis, allograft rejection, arthritis, asthma, lupus, adult respiratory distress syndrome, chronic obstructive pulmonary disease, psoriasis, pancreatitis, peritonitis, burns, ischemia, Behcet's disease, graft versus host disease, inflammatory bowel disease, Crohn's disease, ulcerative colitis, multiple sclerosis, and cachexia.

79. A method of treating an inflammatory condition in a patient comprising administering to said patient a composition comprising an inhibitor of HMGB receptor binding and/or HMGB signaling and an agent that inhibits complement biological activity.

80. The method of claim 79, wherein said composition further comprises a pharmaceutically acceptable carrier.

81. The method of claim 79, wherein said inhibitor of HMGB receptor binding and/or HMGB signaling is an inhibitor of HMGB1 receptor binding.

82. The method of claim 79, wherein said inhibitor of HMGB receptor binding and/or HMGB signaling is selected from the group consisting of an antibody to HMGB or an antigen-binding fragment thereof, an HMGB small molecule antagonist, an antibody to TLR2 or an antigen-binding fragment thereof, a soluble TLR2 polypeptide, a TLR2 small molecule antagonist, an antibody to RAGE or an antigen-binding fragment thereof, a soluble RAGE polypeptide and a RAGE small molecule antagonist.

83. The method of claim 82, wherein said inhibitor of HMGB receptor binding and/or HMGB signaling is an HMGB small molecule antagonist.

84. The method of claim 83, wherein said HMGB small molecule antagonist is an ester of an alpha-ketoalkanoic acid.

85. The method of claim 84, wherein said ester of an alpha-ketoalkanoic acid is an ester of a C3 to C8, straight chain or branched alpha-ketoalkanoic acid.

86. The method of claim 84, wherein said ester of an alpha-ketoalkanoic acid is an ester of pyruvic acid.

87. The method of claim 84, wherein said ester of an alpha-ketoalkanoic acid is selected from the group consisting of an ethyl ester, a propyl ester, a butyl ester, a carboxymethyl ester, an acetoxymethyl ester, a carbethoxymethyl ester and an ethoxymethyl ester.

88. The method of claim 87, wherein said ester of an alpha-ketoalkanoic acid is ethyl pyruvate.

89. The method of claim 83, wherein said HMGB small molecule antagonist is a compound of Formula (I a) or a pharmaceutically acceptable salt thereof: 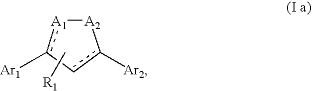
wherein:
Ar₁ and Ar₂ are independently a monocyclic six-member optionally substituted heteroaryl group;
A₁ is ═N— or —NR^{a}— and A₂ is O or S;
R^{a} is H or C1-C6 alkyl;
R₁ is selected from —H, C1-C6 alkyl, phenyl, C1-C6 haloalkyl, halogen, —OH, —OR^{b}, C1-C6 hydroxyalkyl, C1-C6 alkoxyalkyl, —O(C1-C6 haloalkyl), —SH, —SR^{b}, —NO₂, —CN, —NR^{b}CO₂R^{b}, —NR^{b}C(O)R^{b}, —CO₂R^{b}, —C(O)R^{b}, —C(O)N(R^{b})₂, —OC(O)R^{b} and —NR^{b}R^{b}; and
each R^{b} is H or a C1-C6 alkyl group.

90. The method of claim 89 wherein said compound is selected from the group consisting of: 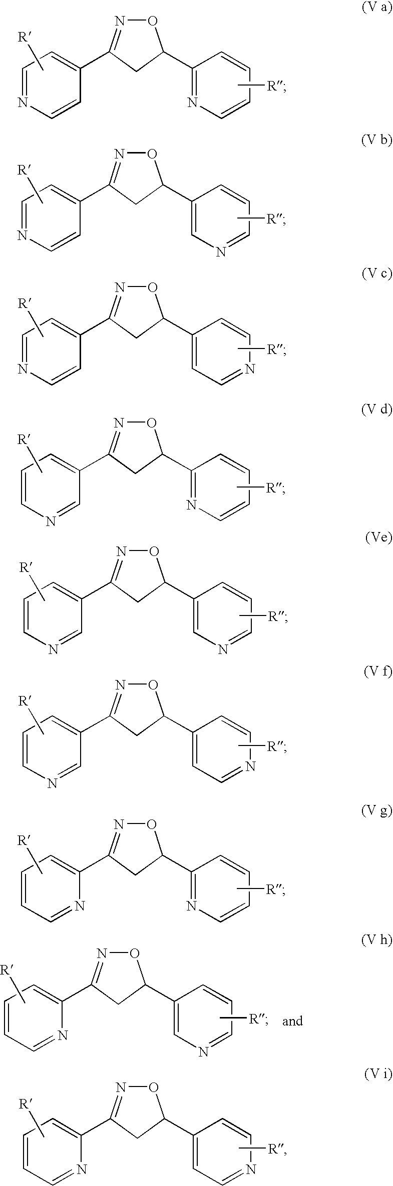
wherein:
R′ and R″ are independently —H, halogen, —NO₂, —CN, C1-C3 alkyl, C1-C3 haloalkyl, C1-C3 hydroxyalkyl, C1-C3 alkoxyalkyl, —N(R^{d})₂, —NR^{d}C(O)R^{d}, or —C(O)N(R^{d})₂.

91. The method of claim 90 wherein said compound is selected from the group consisting of:

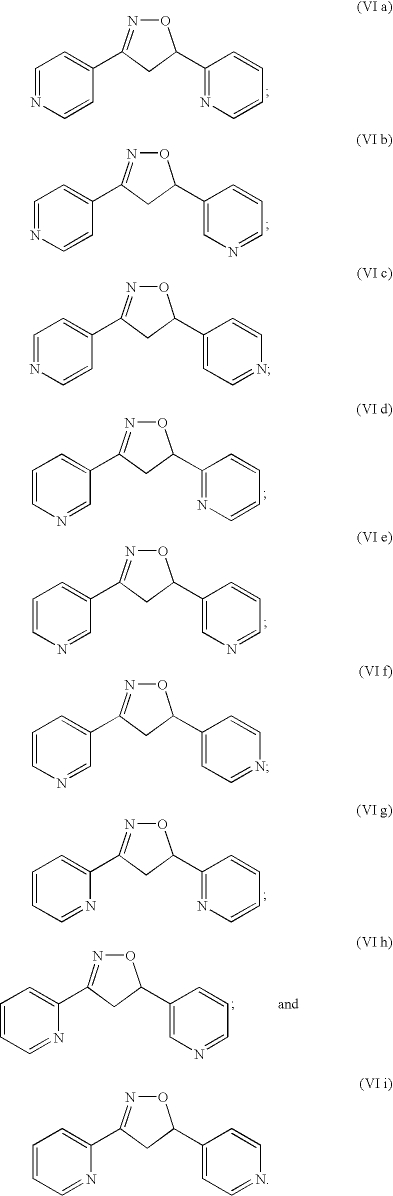

92. The method of claim 91 wherein said compound is represented by Formula (VI d):

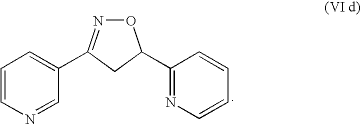

93. The method of claim 92 wherein said compound is represented by Formula (VIf):

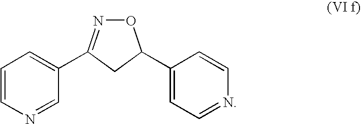

94. The method of claim 79, wherein said agent that inhibits complement biological activity is selected from the group consisting of a C5 inhibitor, a C5a receptor antagonist, a C1 esterase inhibitor, Factor H, Factor I, a soluble complement

95. The method of claim 94, wherein said agent that inhibits complement biological activity is selected from the group consisting of: a C5 inhibitor, a C5a receptor antagonist, a C1 esterase inhibitor, Factor H, Factor I, a soluble complement receptor type 1 (sCR1), a sCR1-sLe(X) cofactor protein, membrane cofactor protein (MCP) or a soluble recombinant form thereof, decay accelerating factor (DAF) or a soluble recombinant form thereof, CD59 or a soluble recombinant form thereof, Compstatin, a chimeric complement inhibitor protein comprising at least two complementary inhibitory domains, and a small molecule antagonist.

96. The method of claim 95, wherein said agent that inhibits complement biological activity is a C5 inhibitor selected from the group consisting of 5G1.1 and h5G1.1-SC.

97. The method of claim 95, wherein said agent that inhibits complement biological activity is a C5a receptor antagonist.

98. The method of claim 97, wherein said C5a receptor antagonist is NGD 2000-1.

99. The method of claim 95, wherein said agent that inhibits complement biological activity is a sCR1.

100. The method of claim 79, wherein said inflammatory condition is selected from the group consisting of sepsis, allograft rejection, arthritis, asthma, lupus, adult respiratory distress syndrome, chronic obstructive pulmonary disease, psoriasis, pancreatitis, peritonitis, burns, ischemia, Behcet's disease, graft versus host disease, inflammatory bowel disease, Crohn's disease, ulcerative colitis, multiple sclerosis, and cachexia.

101. A pharmaceutical composition comprising an agent that inhibits HMGB biological activity and an agent that inhibits complement biological activity.

102. A method of treating an inflammatory condition in a patient comprising administering to said patient a composition comprising an agent that inhibits HMGB biological activity and an agent that inhibits complement biological activity.
